SEQUENCE LISTING (40 sequences)

SEQ ID NO: 1 - protein, 8 aa, synthetic construct
  REGION 1..8: note = Synthetic Polypeptide
SLKRYGGG

SEQ ID NO: 2 - protein, 8 aa, synthetic construct
  REGION 1..8: note = Synthetic Polypeptide
AAFRSRGA

SEQ ID NO: 3 - protein, 8 aa, synthetic construct
  REGION 1..8: note = Synthetic Polypeptide
  REGION 1..2: note = MISC_FEATURE - Xaa can be any amino acid
  REGION 7..8: note = MISC_FEATURE - Xaa can be any amino acid
XXFRFFXX

SEQ ID NO: 4 - protein, 6 aa, synthetic construct
  REGION 1..6: note = Synthetic Polypeptide
QSVGFA

SEQ ID NO: 5 - protein, 7 aa, synthetic construct
  REGION 1..7: note = Synthetic Polypeptide
LGLEGAD

SEQ ID NO: 6 - protein, 4 aa, synthetic construct
  REGION 1..4: note = Synthetic Polypeptide
GPLD

SEQ ID NO: 7 - protein, 6 aa, synthetic construct
  REGION 1..6: note = Synthetic Polypeptide
LGVLIV

SEQ ID NO: 8 - protein, 6 aa, synthetic construct
  REGION 1..6: note = Synthetic Polypeptide
GLVLVA

SEQ ID NO: 9 - protein, 6 aa, synthetic construct
  REGION 1..6: note = Synthetic Polypeptide
PAALVG

SEQ ID NO: 10 - protein, 7 aa, synthetic construct
  REGION 1..7: note = Synthetic Polypeptide
GPAGLAG

SEQ ID NO: 11 - protein, 10 aa, synthetic construct
  REGION 1..10: note = Synthetic Polypeptide
GGPLGVRGKK

SEQ ID NO: 12 - protein, 10 aa, synthetic construct
  REGION 1..10: note = Synthetic Polypeptide
  SITE 3: note = MISC_FEATURE - D amino acid
GGFPRSGGGK

SEQ ID NO: 13 - protein, 14 aa, synthetic construct [sequence omitted]

SEQ ID NO: 14 - protein, 36 aa, synthetic construct
  REGION 1..36: note = Synthetic Polypeptide
ACEQNPIYWARYADWLFTTPLLLLDLALLVDADEGT

SEQ ID NO: 15 - protein, 32 aa, synthetic construct
  REGION 1..32: note = Synthetic Polypeptide
ACEDQNPYWARYADWLFTTPLLLLDLALLVDG

SEQ ID NO: 16 - protein, 30 aa, synthetic construct
  REGION 1..30: note = Synthetic Polypeptide
ACEDQNPYWRAYADLFTPLTLLDLLALWDG

SEQ ID NO: 17 - protein, 27 aa, synthetic construct
  REGION 1..27: note = Synthetic Polypeptide
ACDDQNPWRAYLDLLFPTDTLLLDLLW

SEQ ID NO: 18 - protein, 27 aa, synthetic construct
  REGION 1..27: note = Synthetic Polypeptide
ACEEQNPWRAYLELLFPTETLLLELLW

SEQ ID NO: 19 - protein, 25 aa, synthetic construct
  REGION 1..25: note = Synthetic Polypeptide
ACDDQNPWARYLDWLFPTDTLLLDL

SEQ ID NO: 20 - protein, 24 aa, synthetic construct
  REGION 1..24: note = Synthetic Polypeptide
CDNNNPWRAYLDLLFPTDTLLLDW

SEQ ID NO: 21 - protein, 25 aa, synthetic construct
  REGION 1..25: note = Synthetic Polypeptide
ACEEQNPWARYLEWLFPTETLLLEL

SEQ ID NO: 22 - protein, 24 aa, synthetic construct
  REGION 1..24: note = Synthetic Polypeptide
CEEQQPWAQYLELLFPTETLLLEW

SEQ ID NO: 23 - protein, 24 aa, synthetic construct
  REGION 1..24: note = Synthetic Polypeptide
CEEQQPWRAYLELLFPTETLLLEW

SEQ ID NO: 24 - protein, 24 aa, synthetic construct
  REGION 1..24: note = Synthetic Polypeptide
ACEDQNPWARYADWLFPTTLLLLD

SEQ ID NO: 25 - protein, 24 aa, synthetic construct
  REGION 1..24: note = Synthetic Polypeptide
ACEEQNPWARYAEWLFPTTLLLLE

SEQ ID NO: 26 - protein, 22 aa, synthetic construct
  REGION 1..22: note = Synthetic Polypeptide
ACEDQNPWARYADLLFPTTLAW

SEQ ID NO: 27 - protein, 22 aa, synthetic construct
  REGION 1..22: note = Synthetic Polypeptide
ACEEQNPWARYAELLFPTTLAW

SEQ ID NO: 28 - protein, 34 aa, synthetic construct
  REGION 1..34: note = Synthetic Polypeptide
  SITE 1: note = Modified with acetyl group
  SITE 34: note = Modified with amide group
TEDADVLLALDLLLLPTTFLWDAYRAWYPNQECA

SEQ ID NO: 29 - protein, 37 aa, synthetic construct
  REGION 1..37: note = Synthetic Polypeptide
CDDDDDNPNYWARYANWLFTTPLLLLNGALLVEAEET

SEQ ID NO: 30 - protein, 37 aa, synthetic construct
  REGION 1..37: note = Synthetic Polypeptide
CDDDDDNPNYWARYAPWLFTTPLLLLPGALLVEAEET

SEQ ID NO: 31 - protein, 37 aa, synthetic construct
  REGION 1..37: note = Synthetic Polypeptide
AAEQNPIYWARYADWLFTTPLLLLDLALLVDADEGTC

SEQ ID NO: 32 - protein, 37 aa, synthetic construct; identical to SEQ ID NO: 31
  REGION 1..37: note = Synthetic Polypeptide
  SITE 37: note = Modified with cyanine

SEQ ID NO: 33 - protein, 29 aa, synthetic construct
  REGION 1..29: note = Synthetic Polypeptide
  REGION 28..29: note = MISC_FEATURE - D-amino acids
ACDDQNPWRAYLDLLFPTDTLLLDLLWKK

SEQ ID NO: 34 - protein, 29 aa, synthetic construct
  REGION 1..29: note = Synthetic Polypeptide
  REGION 2..3: note = Modified with cyanine
  SITE 28: note = D-Lysine
  SITE 29: note = D-Lysine
ACDDQNPWRAYLDLLFPTDTLLLDLLWKK

SEQ ID NO: 35 - protein, 32 aa, synthetic construct
  REGION 1..32: note = Synthetic Polypeptide
  SITE 1: note = Modified with azide side chain
  SITE 31: note = D-Lysine
  SITE 32: note = D-Lysine
KGGACDDQNPWRAYLDLLFPTDTLLLDLLWKK

SEQ ID NO: 36 - protein, 32 aa, synthetic construct; identical to SEQ ID NO: 35
  REGION 1..32: note = Synthetic Polypeptide
  SITE 1: note = Modified with azide side chain
  SITE 4: note = D-Alanine
  SITE 5: note = D-Cysteine
  SITE 6..7: note = D-Aspartate
  SITE 8: note = D-Glutamine
  SITE 9: note = D-Asparagine
  SITE 10: note = D-Proline
  SITE 11: note = D-Tryptophan
  SITE 12: note = D-Arginine
  SITE 13: note = D-Alanine
  SITE 14: note = D-Tyrosine
  SITE 15: note = D-Leucine
  SITE 16: note = D-Aspartate
  SITE 17..18: note = D-Leucine
  SITE 19: note = D-Phenylalanine
  SITE 20: note = D-Proline
  SITE 21: note = D-Threonine
  SITE 22: note = D-Aspartate
  SITE 23: note = D-Threonine
  SITE 24..26: note = D-Leucine
  SITE 27: note = D-Aspartate
  SITE 28..29: note = D-Leucine
  SITE 30: note = D-Tryptophan
  SITE 31..32: note = D-Lysine

SEQ ID NO: 37 - protein, 29 aa, synthetic construct
  REGION 1..29: note = Synthetic Polypeptide
  SITE 28..29: note = D-Lysine
ACDDQNPWRAYLKLLFPTKTLLLKLLWKK

SEQ ID NO: 38 - protein, 29 aa, synthetic construct
  REGION 1..29: note = Synthetic Polypeptide
  REGION 2..3: note = Modified with cyanine
  SITE 28..29: note = D-Lysine
ACDDQNPWRAYLKLLFPTKTLLLKLLWKK

SEQ ID NO: 39 - protein, 32 aa, synthetic construct
  REGION 1..32: note = Synthetic Polypeptide
  SITE 1: note = Modified with azide side chain
  SITE 31..32: note = D-Lysine
KGGACDDQNPWRAYLKLLFPTKTLLLKLLWKK

SEQ ID NO: 40 - protein, 32 aa, synthetic construct; identical to SEQ ID NO: 35
  REGION 1..32: note = Synthetic Polypeptide
  SITE 1: note = MISC_FEATURE - Modified with azide side chain
  SITE 4: note = D-Alanine
  SITE 5: note = D-Cysteine
  SITE 6..7: note = D-Aspartate
  SITE 8: note = D-Glutamine
  SITE 9: note = D-Asparagine
  SITE 10: note = D-Proline
  SITE 11: note = D-Tryptophan
  SITE 12: note = D-Arginine
  SITE 13: note = D-Alanine
  SITE 14: note = D-Tyrosine
  SITE 15: note = D-Leucine
  SITE 16: note = D-Aspartate
  SITE 17..18: note = D-Leucine
  SITE 19: note = D-Phenylalanine
  SITE 20: note = D-Proline
  SITE 21: note = D-Threonine
  SITE 22: note = D-Aspartate
  SITE 23: note = D-Threonine
  SITE 24..26: note = D-Leucine
  SITE 27: note = D-Aspartate
  SITE 28..29: note = D-Leucine
  SITE 30: note = D-Tryptophan
  SITE 31..32: note = D-Lysine

(19) United States
(12) Patent Application Publication
(10) Pub. No.: US 2024/0280576 A1
(43) Pub. Date: Aug. 22, 2024

(54) SENSORS FOR DETECTING AND IMAGING OF CANCER METASTASIS

(71) Applicant: Massachusetts Institute of Technology, Cambridge, MA (US)

(72) Inventors: Sangeeta N. Bhatia, Lexington, MA (US); Liangliang Hao, Winchester, MA (US)

(73) Assignee: Massachusetts Institute of Technology, Cambridge, MA (US)

(21) Appl. No.: 18/493,878

(22) Filed: Oct. 25, 2023

Related U.S. Application Data
(63) Continuation of application No. 16/745,748, filed on Jan. 17, 2020, now Pat. No. 11,835,522.
(60) Provisional application No. 62/793,805, filed on Jan. 17, 2019.

(51) Int. Cl. G01N 33/573; G01N 33/534; G01N 33/574

(52) U.S. Cl. CPC G01N 33/573; G01N 33/534; G01N 33/574

(57) ABSTRACT

In some aspects, the disclosure relates to compositions and method for detection, classification, and treatment of cancer. In some embodiments, the disclosure relates to protease imaging sensors comprising a scaffold linked to an enzyme-specific substrate that includes a first detectable marker capable of being released from the prostate protease sensor when exposed to an enzyme present in cancer and a tumor imaging agent comprising a second detectable marker that is linked to the scaffold. In some embodiments, the disclosure relates to methods of monitor progression of a tumor in a subject based upon detection of detectable markers in a sample obtained from a subject who has been administered a protease imaging sensor, upon detection of a tumor imaging agent, or any combination thereof.

### CROSS-REFERENCE TO RELATED APPLICATIONS

This application is a continuation of U.S. application Ser. No. 16/745,748, filed Jan. 17, 2020, which claims the benefit of priority under 35 U.S.C. § 119(e) of U.S. provisional application Ser. No. 62/793,805, filed Jan. 17, 2019, the disclosure of each of which is incorporated by reference herein in its entirety.

### REFERENCE TO AN ELECTRONIC SEQUENCE LISTING

The contents of the electronic sequence listing (M065670441US02-SEQ-FL.xml; Size: 75,763 bytes; and Date of Creation: Oct. 24, 2023) is herein incorporated by reference in its entirety.

### FIELD

The disclosure relates, in some aspects, to improved methods and products associated with detecting, localizing, and monitoring the activity of proteases in vivo or in vitro. These methods and products form the basis of, and may be used as, an ultrasensitive diagnostic platform.

### BACKGROUND

More than 90% of all cancer-related deaths are caused by metastasis. For example, five-year survival rate of colorectal cancer (CRC) patients with large, distant metastases is significantly reduced compared to those with only localized, primary lesions. Although early detection of metastases may save many lives, their small size, multiplicity and ability to invade diverse organs make sensitive detection an elusive goal. Beyond early detection, pretreatment stratification and response assessment are critical to establishing a robust therapeutic strategy.

### SUMMARY

Aspects of the present disclosure provide a protease imaging sensor comprising: (a) a scaffold linked to an enzyme-specific substrate that is attached to a first detectable marker, wherein the first detectable marker is capable of being released from the sensor when exposed to an enzyme, and, (b) a tumor imaging agent comprising a second detectable marker, wherein the tumor imaging agent is linked to the scaffold and wherein the tumor imaging agent does not include a cell penetrating domain.

In some embodiments, the tumor imaging agent further comprises a pH low insertion peptide.

In some embodiments, the scaffold comprises a protein, a polymer, or a nanoparticle. The protein, polymer, or nanoparticle may be greater than about 5 nm in diameter.

In some embodiments, the scaffold comprises a multi-arm polyethylene glycol molecule (multi-arm PEG). In some embodiments, the multi-arm PEG comprises 2-20 arms. In some embodiments, the multi-arm PEG comprises 8 arms. In some embodiments, the multi-arm PEG has a total diameter between 5 nm and 20 nm, optionally wherein the multi-arm PEG has a total diameter of about 15 nm.

In some embodiments, the scaffold comprises an iron oxide nanoparticle (IONP), optionally wherein the IONP is between about 10 nm and about 20 nm in size.

In some embodiments, each enzyme-specific substrate comprises a cancer substrate, optionally wherein the cancer substrate is cleaved by an enzyme associated with colorectal cancer (CRC). In some embodiments, the cancer substrate is a cancer metastasis substrate, optionally wherein the cancer metastasis substrate is cleaved by an enzyme associated with colorectal cancer metastasis.

In some embodiments, the first detectable marker comprises a peptide, a nucleic acid, a small molecule, a fluorophore, a carbohydrate, a particle, a radiolabel, a MRI-active compound, a ligand encoded reporter, or a isotope coded reporter molecule (iCORE).

In some embodiments, the second detectable marker comprises a radiolabel and is detectable by positron emission tomography or computerized tomography. In some embodiments, the radiolabel is selected from the group consisting of ⁶⁴Cu, Gd(DOTA), ²⁰¹Tl, ^{99m}Tc, ¹⁸F-2-deoxyfluoroglucose (FDG), (18)F-fluoride, gadodiamide, radioisotopes of Pb(II), ¹¹¹In, and ⁸⁹Zr. In some embodiments, the second detectable marker comprises a metal chelator selected from the group consisting of 1,4,7-Triazacyclononane-1,4,7-triacetic acid (NOTA), 1,4,7,10-tetraazacyclododecane-N,N′,N″,N′″-tetraacetic acid (DOTA), Diethylenetriaminepentaacetic Anhydride (DTPA), 1,4,8,11-tetraazacyclotetradecane-1,4,8,11-tetraacetic acid (TETA), and deferoxamine (e.g., for 89Zr).

In some embodiments, the pH low insertion peptide is selected from the group consisting of SEQ ID NOs: 14-40. In some embodiments, the pH low insertion peptide comprises a D-amino acid, an azide side chain, and/or cyanine. In some embodiments, the tumor imaging peptide is N-terminally linked to the scaffold.

In some embodiments, the scaffold comprises a single enzyme-specific substrate, a single tumor imaging peptide, or a combination thereof. In some embodiments, the scaffold comprises multiple enzyme-specific substrates, multiple tumor imaging peptides, or a combination thereof. In some embodiments, the ratio of the number of enzyme-specific substrates to the number of tumor imaging peptides is 1:1.

Another aspect of the present disclosure provides a method for detecting a tumor in a subject, the method comprising: (a) administering to a subject a protease imaging sensor, wherein the protease imaging sensor comprises (i) a scaffold linked to an enzyme-specific substrate that is attached to a first detectable marker, wherein the first detectable marker is capable of being released from the sensor when exposed to a cancer-associated enzyme at a site within the subject, and (ii) a tumor imaging peptide comprising a second detectable marker, wherein the tumor imaging peptide is linked to the scaffold and wherein the tumor imaging agent does not include a cell penetrating domain; and (b) detecting in a biological sample obtained from the subject the first detectable marker, wherein detection of the first detectable marker in the biological sample is indicative of the subject having a tumor and/or detecting in the subject the second detectable marker, wherein detection of the second detectable marker indicates the site of exposure to the cancer-associated enzyme.

In some embodiments, the tumor imaging peptide comprises a pH low insertion peptide.

In some embodiments, the method comprises detecting the first detectable marker and detecting the second detectable marker.

In some embodiments, the biological sample is not derived from the site of exposure to the cancer-associated enzyme, optionally wherein the sample is a urine sample, blood sample, or tissue sample.

In some embodiments, the site of exposure to the cancer-associated enzyme is a site of metastasis. In some embodiments, the site of metastasis is selected from the group consisting of lung, liver, or heart.

In some embodiments, the method further comprises quantifying the amount of the second detectable marker.

In some embodiments, the detecting of the first detectable marker comprises a method selected from mass spectrometry, PCR analysis, DNA microarray, fluorescence analysis, a capture assay (e.g., ELISA), optical imaging, magnetic resonance imaging (MRI), positron emission tomography (PET) imaging, computerized tomography (CT) imaging, intraoperative imaging or any combination thereof.

In some embodiments, the detecting of the second detectable marker comprises a method selected from fluorescence analysis, optical imaging, magnetic resonance imaging (MRI), positron emission tomography (PET) imaging, computerized tomography (CT) imaging, intraoperative imaging, or any combination thereof.

In some embodiments, the subject is suspected of having, at risk for, or has cancer, optionally wherein the cancer is colorectal cancer.

In some embodiments, the subject has been administered a therapeutic agent.

In some embodiments, the method further comprises classifying a cancer as metastatic or non-metastatic.

Another aspect of the present disclosure provides a method for monitoring tumor progression in a subject, the method comprising: (a) administering to a subject having a tumor a protease imaging sensor, wherein the protease imaging sensor comprises (i) a scaffold linked to an enzyme-specific substrate that is attached to a first detectable marker, wherein the first detectable marker is capable of being released from the sensor when exposed to a cancer-associated enzyme at a site within the subject, and (ii) a tumor imaging peptide comprising a second detectable marker, wherein the tumor imaging peptide is linked to the scaffold and wherein the tumor imaging agent does not include a cell penetrating domain; (b) detecting in a biological sample obtained from the subject the first detectable marker, wherein detection of the first detectable marker in the biological sample is indicative of the subject having a tumor and/or detecting in the subject the second detectable marker, wherein detection of the second detectable marker indicates the site of exposure to the cancer-associated enzyme; and (c) repeating (a) and (b) at least once, thereby monitoring tumor progression in the subject.

In some embodiments, the subject has been administered a first therapeutic agent, a first therapeutic intervention has been performed on the subject, or a combination thereof.

In some embodiments, the method comprises detecting the first detectable marker and detecting the second detectable marker.

In some embodiments, the tumor imaging peptide comprises a pH low insertion peptide.

In some embodiments, the biological sample is not derived from the site of exposure to the cancer-associated enzyme, optionally wherein the sample is a urine sample, blood sample, or tissue sample.

In some embodiments, the site of exposure to the cancer-associated enzyme is a site of metastasis.

In some embodiments, the site of metastasis is selected from the group consisting of lung, liver, or heart.

In some embodiments, the method further comprises quantifying the amount of the second detectable marker.

In some embodiments, the detecting of the first detectable marker comprises a method selected from mass spectrometry, PCR analysis, DNA microarray, fluorescence analysis, a capture assay (e.g., ELISA), optical imaging, magnetic resonance imaging (MRI), positron emission tomography (PET) imaging, computerized tomography (CT) imaging, intraoperative imaging or any combination thereof.

In some embodiments, the detecting of the second detectable marker comprises a method selected from fluorescence analysis, optical imaging, magnetic resonance imaging (MRI), positron emission tomography (PET) imaging, computerized tomography (CT) imaging, intraoperative imaging, or any combination thereof.

In some embodiments, the subject has colorectal cancer.

In some embodiments, the method further comprises classifying a tumor as progressing, in remission, or stable.

The details of one or more embodiments of the invention are set forth in the description below. Other features or advantages of the present invention will be apparent from the following drawings and detailed description of several embodiments, and also from the appended claims.

### BRIEF DESCRIPTION OF DRAWINGS

FIG. 1 shows protease-responsive imaging sensors for detection and imaging of cancer metastasis. PRISM (I) targets acidic TME. Activation of PRISM by metastasis-specific protease activity triggers (II) release of synthetic biomarker into urine for sensitive detection (III). Tumor insertion enables cancer specific imaging signal (IV).

FIGS. 2A-2J show that PRISM detects CRC liver metastasis in a pre-clinical immunocompetent mouse model. FIG. 2A shows a structure of PRISM built on multivalent polyethylene glycol (PEG) scaffolds to carry two functional components. FIG. 2B shows that acidic pH specificity was demonstrated in MMP9-secreting MC26 cells at pH 6.5, with substantially higher cell accumulation than that observed at pH 7.4. FIG. 2C shows a typical in vivo experimental time line. FIG. 2D shows histology of sham (left) and tumor-bearing mice (right). FIG. 2E is a histological image showing the presence of metastasis in the liver. FIG. 2F is a histological image showing the presence of metastasis in the liver. FIG. 2G shows a comparison of urinary signals generated by two sets of sensors bearing pHLIP or its non-targeting counterpart (NT-pHLIP) (SEQ ID NOS: 35 and 39). FIG. 2H shows IHC staining of MMP9 in sham (right) and tumor-bearing mice (left). FIG. 2I shows Cy7-labeled PRISM specifically accumulated in metastasis in liver reflected by the fluorescent signal from the dye-labeled peptides (SEQ ID NOS: 34 and 38). The left image shows localization of pHLIP. The right image shows localization of NT-pHLIP. FIG. 2J shows quantification of fluorescence intensity of pHLIP and NT-pHLIP along the dotted lines shown in FIG. 2I.

FIGS. 3A-3H show ⁶⁴Cu-labeled PRISM detects CRC liver metastasis. FIG. 3A show radioactive PRISM for PET/CT imaging were constructed through chelation of radioactive isotope ⁶⁴Cu through metal chelator NOTA conjugated on the cysteine of pHLIP peptide (SEQ ID NO: 35). FIG. 3B show representative images of healthy mice vs. CLM mice administered with ¹⁸F-FDG and radioactive PRISM (carrying SEQ ID NO: 35) (n=5 mice per group). FIG. 3C show relative reporter concentrations measured in the urine of healthy mice vs. CLM mice after application of PRISM (n=5 mice per group; ±SEM; Student's t-test, two-tailed, ***P<0.001, *P<0.05). FIG. 3D show urinary reporters quantification and transverse images of tumor progression in CLM mice administered with radioactive PRISM over time (n=5 mice per group).

FIG. 3E shows that the standard uptake value in the major organs as indicated were comparable against the conventional PET tracer. FIG. 3F shows that similar tumor versus liver ratios were observed in mice imaged with ¹⁸F-FDG and Cu-PRISM. FIG. 3G shows that the PRISM sensors were colocalized with abnormal physiologic conditions such as hypoxia. FIG. 3H shows that the majority of detected MMP9 protein expression overlapped with FAM-staining that marks PRISM sensors in the tumor, presumably bound to tissue section cells via membrane insertion of pHILP.

FIGS. 4A-4K show PRISM for detection and imaging in CRC lung metastasis model. FIG. 4A shows a typical in vivo experimental time line. FIG. 4B shows histopathology staining of lung sections from MC26 injected tumor bearing mouse and control mouse injected with saline. FIG. 4C shows urinary reporter level quantified in tumor-beading and control mice as tumor progressed. FIG. 4D shows (i-ii) PET/CT images of healthy or mice with CRC lung metastasis visualized by ¹⁸F-FDG; (iii-iv) ⁶⁴Cu-PRISM (carrying SEQ ID NO: 35). FIG. 4E shows relative SUV quantification of images in FIG. 4D. FIG. 4F shows relative SUV quantification of images in FIG. 4E. FIG. 4G shows relative SUV quantification of heart uptake in tumor bearing mice imaged with FDG and PRISM, respectively. FIG. 4H shows relative SUV quantification of lung/liver ratio in sham or tumor bearing mice imaged with PRISM. FIG. 4I shows IHC and immunofluorescence staining on tissue sections from tumor bearing mice. FIG. 4J shows the high-throughput cryo-fluorescence tomography on whole animals (healthy control or lung tumor-bearing) after systemic administration of PRISM sensors. The localization of FAM fluorescence signal exhibits a pattern that was perfectly aligned with corresponding anatomical tumors that had formed throughout the lung. FIG. 4K shows that PRISM sensors were largely colocalized with MMP9, which present at elevated levels in the lung tumors.

FIGS. 5A-5G show PRISM for chemotherapy treatment monitoring in CRC liver metastasis model. FIG. 5A shows a typical in vivo experimental time line. FIG. 5B shows relative reporter concentrations measured in the urine of CLM mice with or without 5-FU treatment (n=5 mice per group; ±SEM; Student's 1-test, two-tailed, ***P<0.001, ****P<0.0001). FIGS. 5C-5F show representative images of CLM mice with or without 5-FU treatment imaged with radioactive PRISM (carrying SEQ ID NO: 35) at 2 (FIGS. 5C and 5E) or 4 weeks (FIGS. 5D and 5F) after tumor inoculation (n=5 mice per group). FIG. 5G shows quantification of metastasis/liver ratio, indicating tumor progression in CLM mice administered with radioactive PRISM (n=5 mice per group).

FIGS. 6A-6G show PRISM for treatment monitoring in CRC lung metastasis model. FIG. 6A shows a typical in vivo experimental time line. FIG. 6B shows relative reporter concentrations measured in the urine of tumor bearing mice with or without 5-FU treatment (n=5 mice per group; ±SEM; Student's 1-test, two-tailed, ***P<0.001, ****P<0.0001). FIGS. 6C-6F show representative images of CLM mice with or without 5-FU treatment imaged with radioactive PRISM (carrying SEQ ID NO: 35) at 2 (FIGS. 6C and 6E) or 4 weeks (FIGS. 6D and 6F) after tumor inoculation (n=5 mice per group). FIG. 6G shows quantification of lung/liver ratio, indicating tumor progression in tumor bearing mice administered with radioactive PRISM (n=5 mice per group).

### DETAILED DESCRIPTION

The technology described herein allows for detection, including early detection and precise imaging of tumors. The present disclosure is based, at least in part, on the unexpected results demonstrating that a protease imaging sensor comprising (i) an enzyme-specific substrate with a first detectable marker that is capable of being released from the sensor upon exposure to an enzyme and (ii) a tumor imaging agent with a second detectable marker can be used to detect tumors with aberrant protease activity and localize to sites of metastasis in a noninvasive manner.

Reliable evidence of disseminated disease at the time of primary detection is consistent with a poor prognosis. For example, though hepatic resection is widely accepted as the optimal modality for potentially curing patients, only 10% of patients with CRC liver metastases at primary diagnosis are candidates for this intervention (Hess et al., Cancer. 2006 Apr. 1; 106(7): 1624-33) Thus far, disseminated disease is still largely treated with 5-FU/Leucovorin, a standard chemotherapy which lacks curative potential due to inadequate access to sites of metastasis and emergence of resistance (Martini et al., World J Gastroenterol. 2017 Jul. 14; 23(26):4675-4688). To increase the effectiveness of treatments, intensive surveillance protocols including medical imaging and monitoring for cancer biomarkers in blood are implemented following primary resection to detect early stage metastases (Tsikitis et al., J Clin Oncol. 2009 Aug. 1; 27(22):3671-6). However, the need for sensitive detection places a unique burden on the development of next-generation diagnostics. The small size, multiplicity, and low tumor-to-organ contrast of liver metastases render current imaging platforms (e.g. ultrasound, CT) relatively insensitive to early diseases (e.g., with standard CT scan, only 50% of 1-2 cm nodules are reliably detected, and nodules <5 mm are undetectable (Schima et al., Cancer Imaging. 2005 Nov. 23; 5 Spec No A:S149-56). Similar challenges exist for blood biomarker detection, where solid tumors could potentially remain undetectable for 10-12 years and reach spherical diameters >2.5 cm before blood biomarker levels can indicate the disease (Hori et al., Sci Transl Med. 2011 Nov. 16; 3(109):109ra116; Lutz et al., PLOS Med. 2008 Aug. 19; 5(8):c170). Moreover, inconsistencies between the mutation status estimated using blood versus tumor DNA samples also interfere with early detection and diagnosis. Beyond initial diagnosis, disease stratification is also critical to design a robust treatment strategy. Recently, for example, improvements in patient survival and reduced recurrence in CRC patients have been attributed to increased incidence of liver resection in a selected group of patients bearing a maximum four focal liver lesions (Tsikitis et al., J Clin Oncol. 2009 Aug. 1; 27(22):3671-6; Blesa et al., Curr Oncol. 2009 September; 16(5):76-80).

In some embodiments, the multi-functional protease imaging sensors integrate advances in nanoscale materials, tumor-insertion peptides, and synthetic biomarkers in order to treat metastases and sensitively detect their response beyond clinical thresholds. Unlike traditional shotgun proteomic methods which seek to identify biomarkers based on the abundance of proteins, the activity-based sensor (e.g., activity-based nanosensor, ABN) approach is based on the application of synthetic peptides to monitor protease activity. Thus, it circumvents challenges associated with shotgun methods including low target protein concentrations, low signal-to-noise ratios due to matrix complexity, and the need for rigorous protocol validation (Picotti et al., Nature methods 2012, 9, 555). In some embodiments, the protease imaging sensors comprise nanomaterial pharmacokinetics (e.g., urinary secretion) and bio-orthogonality (e.g., synthetic reporters not present in living systems). Without being bound by a particular theory, these degrees of precision are not readily amenable to endogenous biomarkers and may provide the ability to detect metastases earlier than clinically used diagnostics.

Furthermore, the key question in development of a novel therapy regimen, especially immunotherapies, remains whether it is possible to predict the outcome of therapy, based not only on the presence of cytotoxic T-cells but also on their interaction with and dynamic changes in the tumor microenvironment over time (Rashidian et al., The Journal of experimental medicine 2017, 214, 2243; Helfen et al., Journal of nuclear medicine: official publication, Society of Nuclear Medicine 2018, 59, 183). Standard PET imaging with metabolic tracers has been widely used to assess response to traditional therapy but is limited in cancer immunotherapy due to a lack of ability to accurately track changes in tumor microenvironment (TME) (Longo et al., Cancer research 2016, 76, 6463; Larimer et al., Cancer research 2017, 77, 2318). When immune cells infiltrate tumors, they can cause expansion of tumor volume or induce new detectable lesions that are indistinguishable from tumor progression (Rashidian et al., Proceedings of the National Academy of Sciences of the United States of America 2015, 112, 6146). As an extension of the methods described herein, without being bound by a particular theory, one could detect and track changes in TME noninvasively via pathologic acidosis targeting. Such level of precision would allow one to follow and visualize therapeutic responses longitudinally and also to predict outcomes. Patients may then be stratified into responders and non-responders during the course of immunotherapy, such that decisions to continue or terminate therapy might be refined in case of an equivocal response. This level of noninvasive monitoring could therefore change how therapies are applied and assessed, to the benefit of many patients.

The protease imaging sensors of the present disclosure address the foregoing challenges, in part, by allowing for robust tumor detection and providing precise localization of metastases in disseminated disease models that recapitulate important features of human disease. In some embodiments, when the first detectable marker is detected (e.g. in the urine), precise anatomical information such as size, shape, position of tumors, and their relationship with adjacent structures may be determined using the second detectable marker for clinical therapy response evaluation and treatment plan reaffirmation. The protease imaging sensors of the present disclosure may be useful in the sensitive detection of cancer metastases and in increasing efficacy of treatments. For example, in some embodiments, the protease imaging sensors harness metastasis-specific proteases as triggers to release urinary biomarkers for sensitive detection and imaging of tumor metastases.

### Scaffolds

The protease imaging sensor (e.g., protease imaging sensor) comprises a modular structure having a scaffold linked to an enzyme-specific substrate that is attached to a first detectable marker and a tumor imaging agent. A modular structure, as used herein, refers to a molecule having multiple domains.

The scaffold may include a single type of substrate, such as, a single type of enzyme-specific substrate, or it may include multiple types of different substrates. For instance each scaffold may include a single (e.g., 1) type of substrate or it may include 2-1,000 different substrates, or any integer therebetween. Alternatively, each scaffold may include greater than 1,000 different substrates. Multiple copies of the protease imaging sensor are administered to the subject. In some embodiments, a composition comprising a plurality of different sensors may be administered to a subject to determine whether multiple enzymes and/or substrates are present. In that instance, the plurality of different sensors includes a plurality of first detectable markers, such that each substrate is associated with a first detectable marker.

The scaffold may serve as the core of the sensor. A purpose of the scaffold is to serve as a platform for the substrate and enhance delivery of the sensor to the subject. As such, the scaffold can be any material or size as long as it can enhance delivery and/or accumulation of the sensors to the subject. Preferably, the scaffold material is non-immunogenic, i.e. does not provoke an immune response in the body of the subject to which it will be administered. Non-limiting examples of scaffolds, include, for instance, compounds that cause active targeting to tissue, cells or molecules, microparticles, nanoparticles, aptamers, peptides (RGD, iRGD, LyP-1, CREKA, etc.), proteins, nucleic acids, polysaccharides, polymers, antibodies or antibody fragments (e.g., herceptin, cetuximab, panitumumab, etc.) and small molecules (e.g., erlotinib, gefitinib, sorafenib, etc.).

In some aspects, the disclosure relates to the discovery that delivery to a subject is enhanced by sensors having certain polymer scaffolds (e.g., poly(ethylene glycol) (PEG) scaffolds). Polyethylene glycol (PEG), also known as poly(oxyethylene) glycol, is a condensation polymer of ethylene oxide and water having the general chemical formula HO(CH₂CH₂O)[n]H. Generally, a PEG polymer can range in size from about 2 subunits (e.g., ethylene oxide molecules) to about 50,000 subunits (e.g., ethylene oxide molecules. In some embodiments, a PEG polymer comprises between 2 and 10,000 subunits (e.g., ethylene oxide molecules).

A PEG polymer can be linear or multi-armed (e.g., dendrimeric, branched geometry, star geometry, etc.). In some embodiments, a scaffold comprises a linear PEG polymer. In some embodiments, a scaffold comprises a multi-arm PEG polymer. In some embodiments, a multi-arm PEG polymer comprises between 2 and 20 arms. In some embodiments, a multi-arm PEG polymer comprises at least 1, 2, 3, 4, 5, 6, 7, 8, 9, 10, 15, 20, 30, 40, 50, 60, 70, 80, 90, or 100 arms. In some embodiments, a multi-arm PEG polymer comprises 8 arms. Multi-arm and dendrimeric scaffolds are generally described, for example by Madaan et al. J Pharm Bioallied Sci. 2014 6(3): 139-150.

Additional polymers include, but are not limited to: polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate, poly vinyl chloride and polystyrene.

Examples of non-biodegradable polymers include ethylene vinyl acetate, poly(meth) acrylic acid, polyamides, copolymers and mixtures thereof.

Examples of biodegradable polymers include synthetic polymers such as polymers of lactic acid and glycolic acid, polyanhydrides, poly(ortho)esters, polyurethanes, poly(butic acid), poly(valeric acid), poly(caprolactone), poly(hydroxybutyrate), poly(lactide-co-glycolide) and poly(lactide-co-caprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion. The foregoing materials may be used alone, as physical mixtures (blends), or as co-polymers. In some embodiments the polymers are polyesters, polyanhydrides, polystyrenes, polylactic acid, polyglycolic acid, and copolymers of lactic and glycolic acid and blends thereof.

PVP is a non-ionogenic, hydrophilic polymer having a mean molecular weight ranging from approximately 10,000 to 700,000 and the chemical formula (C₆H₉NO)[n]. PVP is also known as poly [1-(2-oxo-1-pyrrolidinyl)ethylene], Povidone™, Polyvidone™, RP 143™, Kollidon™, Peregal ST™, Periston™, Plasdone™, Plasmosan™, Protagent™, Subtosan™, and Vinisil™. PVP is non-toxic, highly hygroscopic and readily dissolves in water or organic solvents.

Polyvinyl alcohol (PVA) is a polymer prepared from polyvinyl acetates by replacement of the acetate groups with hydroxyl groups and has the formula (CH₂CHOH)[n]. Most polyvinyl alcohols are soluble in water.

PEG, PVA and PVP are commercially available from chemical suppliers such as the Sigma Chemical Company (St. Louis, Mo.).

In certain embodiments the particles may comprise poly(lactic-co-glycolic acid) (PLGA).

In some embodiments, a scaffold (e.g., a polymer scaffold, such as a PEG scaffold) has a molecular weight equal to or greater than 40 kDa. In some embodiments, a scaffold is a nanoparticle (e.g., an iron oxide nanoparticle, IONP) that is between 10 nm and 50 nm in diameter (e.g. having an average particle size between 10 nm and 50 nm, inclusive). In some embodiments, a scaffold is a high molecular weight protein, for example an Fc domain of an antibody.

As used herein the term “particle” includes nanoparticles as well as microparticles. Nanoparticles are defined as particles of less than 1.0 μm in diameter. A preparation of nanoparticles includes particles having an average particle size of less than 1.0 μm in diameter. Microparticles are particles of greater than 1.0 μm in diameter but less than 1 mm. A preparation of microparticles includes particles having an average particle size of greater than 1.0 μm in diameter. The microparticles may therefore have a diameter of at least 5, at least 10, at least 25, at least 50, or at least 75 microns, including sizes in ranges of 5-10 microns, 5-15 microns, 5-20 microns, 5-30 microns, 5-40 microns, or 5-50 microns. A composition of particles may have heterogeneous size distributions ranging from 10 nm to mm sizes. In some embodiments the diameter is about 5 nm to about 500 nm. In other embodiments, the diameter is about 100 nm to about 200 nm. In other embodiment, the diameter is about 10 nm to about 100 nm.

The particles may be composed of a variety of materials including iron, ceramic, metallic, natural polymer materials (including lipids, sugars, chitosan, hyaluronic acid, etc.), synthetic polymer materials (including poly-lactide-coglycolide, poly-glycerol sebacate, etc.), and non-polymer materials, or combinations thereof.

The particles may be composed in whole or in part of polymers or non-polymer materials. Non-polymer materials, for example, may be employed in the preparation of the particles. Exemplary materials include alumina, calcium carbonate, calcium sulfate, calcium phosphosilicate, sodium phosphate, calcium aluminate, calcium phosphate, hydroxyapatite, tricalcium phosphate, dicalcium phosphate, tricalcium phosphate, tetracalcium phosphate, amorphous calcium phosphate, octacalcium phosphate, and silicates. In certain embodiments the particles may comprise a calcium salt such as calcium carbonate, a zirconium salt such as zirconium dioxide, a zinc salt such as zinc oxide, a magnesium salt such as magnesium silicate, a silicon salt such as silicon dioxide or a titanium salt such as titanium oxide or titanium dioxide. A number of biodegradable and non-biodegradable biocompatible polymers are known in the field of polymeric biomaterials, controlled drug release and tissue engineering (see, for example, U.S. Pat. Nos. 6,123,727; 5,804,178; 5,770,417; 5,736,372; 5,716,404 to Vacanti; U.S. Pat. Nos. 6,095,148; 5,837,752 to Shastri; U.S. Pat. No. 5,902,599 to Anseth; U.S. Pat. Nos. 5,696,175; 5,514,378; 5,512,600 to Mikos; U.S. Pat. No. 5,399,665 to Barrera; U.S. Pat. No. 5,019,379 to Domb; U.S. Pat. No. 5,010,167 to Ron; U.S. Pat. No. 4,946,929 to d′Amore; and U.S. Pat. Nos. 4,806,621; 4,638,045 to Kohn; see also Langer, Acc. Chem. Res. 33:94, 2000; Langer, J. Control Release 62:7, 1999; and Uhrich et al., Chem. Rev. 99:3181, 1999; all of which are incorporated herein by reference).

The scaffold may be composed of inorganic materials. Inorganic materials include, for instance, magnetic materials, conductive materials, and semiconductor materials. In some embodiments, the scaffold is composed of an organic material.

In some embodiments, the particles are porous. A porous particle can be a particle having one or more channels that extend from its outer surface into the core of the particle. In some embodiments, the channel may extend through the particle such that its ends are both located at the surface of the particle. These channels are typically formed during synthesis of the particle by inclusion followed by removal of a channel forming reagent in the particle. The size of the pores may depend upon the size of the particle. In certain embodiments, the pores have a diameter of less than 15 microns, less than 10 microns, less than 7.5 microns, less than 5 microns, less than 2.5 microns, less than 1 micron, less than 0.5 microns, or less than 0.1 microns. The degree of porosity in porous particles may range from greater than 0 to less than 100% of the particle volume. The degree of porosity may be less than 1%, less than 5%, less than 10%, less than 15%, less than 20%, less than 25%, less than 30%, less than 35%, less than 40%, less than 45%, or less than 50%. The degree of porosity can be determined in a number of ways. For example, the degree of porosity can be determined based on the synthesis protocol of the scaffolds (e.g., based on the volume of the aqueous solution or other channel-forming reagent) or by microscopic inspection of the scaffolds post-synthesis.

The plurality of particles may be homogeneous for one or more parameters or characteristics. A plurality that is homogeneous for a given parameter, in some instances, means that particles within the plurality deviate from each other no more than about +/−10%, preferably no more than about +/−5%, and most preferably no more than about +/−1% of a given quantitative measure of the parameter. As an example, the particles may be homogeneously porous. This means that the degree of porosity within the particles of the plurality differs by not more than +/−10% of the average porosity. In other instances, a plurality that is homogeneous means that all the particles in the plurality were treated or processed in the same manner, including for example exposure to the same agent regardless of whether every particle ultimately has all the same properties. In still other embodiments, a plurality that is homogeneous means that at least 80%, preferably at least 90%, and more preferably at least 95% of particles are identical for a given parameter.

The plurality of particles may be heterogeneous for one or more parameters or characteristics. A plurality that is heterogeneous for a given parameter, in some instances, means that particles within the plurality deviate from the average by more than about +/−10%, including more than about +/−20%. Heterogeneous particles may differ with respect to a number of parameters including their size or diameter, their shape, their composition, their surface charge, their degradation profile, whether and what type of agent is comprised by the particle, the location of such agent (e.g., on the surface or internally), the number of agents comprised by the particle, etc. The disclosure contemplates separate synthesis of various types of particles which are then combined in any one of a number of pre-determined ratios prior to contact with the sample. As an example, in one embodiment, the particles may be homogeneous with respect to shape (e.g., at least 95% are spherical in shape) but may be heterogeneous with respect to size, degradation profile and/or agent comprised therein.

Particle size, shape and release kinetics can also be controlled by adjusting the particle formation conditions. For example, particle formation conditions can be optimized to produce smaller or larger particles, or the overall incubation time or incubation temperature can be increased, resulting in particles which have prolonged release kinetics.

The particles may also be coated with one or more stabilizing substances, which may be particularly useful for long term depoting with parenteral administration or for oral delivery by allowing passage of the particles through the stomach or gut without dissolution. For example, particles intended for oral delivery may be stabilized with a coating of a substance such as mucin, a secretion containing mucopolysaccharides produced by the goblet cells of the intestine, the submaxillary glands, and other mucous glandular cells.

To enhance delivery the particles may be incorporated, for instance, into liposomes, virosomes, cationic lipids or other lipid based structures. The term “cationic lipid” refers to lipids which carry a net positive charge at physiological pH. Such lipids include, but are not limited to, DODAC, DOTMA, DDAB, DOTAP, DC-Chol and DMRIE. Additionally, a number of commercial preparations of cationic lipids are available. These include, for example, LIPOFECTIN® (commercially available cationic liposomes comprising DOTMA and DOPE, from GIBCO/BRL, Grand Island, N.Y., USA); LIPOFECTAMINE® (commercially available cationic liposomes comprising DOSPA and DOPE, from GIBCO/BRL); and TRANSFECTAM® (commercially available cationic lipids comprising DOGS in ethanol from Promega Corp., Madison, Wis., USA). A variety of methods are available for preparing liposomes e.g., U.S. Pat. Nos. 4,186,183, 4,217,344, 4,235,871, 4,261,975, 4,485,054, 4,501,728, 4,774,085, 4,837,028, 4,946,787; and PCT Publication No. WO 91/17424. The particles may also be composed in whole or in part of GRAS components. i.e., ingredients are those that are Generally Regarded As Safe (GRAS) by the US FDA. GRAS components useful as particle material include non-degradable food based particles such as cellulose.

### Substrates

The enzyme-specific substrate is a portion of the modular structure that is connected to the scaffold. A substrate, as used herein, is the portion of the modular structure that promotes the enzymatic reaction in the subject, causing the release of a detectable marker. The substrate typically comprises an enzyme-sensitive portion (e.g., protease substrate) linked to a detectable marker.

The substrate is dependent on enzymes that are active in a specific disease state (e.g., cancer). For instance, tumors are associated with a specific set of enzymes. A sensor is designed with one or more substrates that match those of the enzymes expressed by the tumor, by the subject in response to the cancer or by other diseased tissue. Alternatively, the substrate may be associated with enzymes that are ordinarily present but are absent in a particular disease state. In this example, a disease state would be associated with a lack of signal associated with the enzyme, or reduced levels of signal compared to a normal reference.

An enzyme, as used herein refers to any of numerous proteins produced in living cells that accelerate or catalyze the metabolic processes of an organism. Enzymes act on substrates. The substrate binds to the enzyme at a location called the active site just before the reaction catalyzed by the enzyme takes place. Enzymes include but are not limited to proteases, glycosidases, lipases, heparinases, phosphatases.

The substrate may be optimized to provide both high catalytic activity (or other enzymatic activity) for specified target enzymes but to also release optimized detectable markers for detection. Patient outcome depends on the phenotype of individual diseases at the molecular level, and this is often reflected in expression of enzymes. The recent explosion of bioinformatics has facilitated exploration of complex patterns of gene expression in human tissues (Fodor S. P. A., Massively parallel genomics. Science 277, 393-395 (1997)). Sophisticated computer algorithms have been recently developed capable of molecular diagnosis of tumors using the immense data sets generated by expression profiling (Khan J, Wei J S, Ringner M, Saal L H, Ladanyi M, Westermann F, et al. Classification and diagnostic prediction of cancers using gene expression profiling and artificial neural networks. Nat Med 2001; 7:673-679). This information can be accessed in order to identify enzymes and substrates associated with specific diseases. Based on this information the skilled artisan can identify appropriate enzyme or substrates to incorporate into the sensor.

In some embodiments, an enzyme-specific substrate comprises a substrate for a protease (e.g., an amino acid sequence that is cleaved by a protease). In some embodiments, the protease substrate is a substrate of a disease-associated enzyme. Examples of enzymes that are associated with disease in a subject include serine proteases, cysteine proteases, threonine proteases, aspartic proteases, glutamic proteases, metalloproteases, etc. Examples of substrates for disease-associated enzymes include but are not limited to SLKRYGGG (SEQ ID NO: 1; plasma kallikrein), AAFRSRGA (SEQ ID NO: 2; kallikrein 1), xxFRFFxx (SEQ ID NO: 3; cathepsin B), QSVGFA (SEQ ID NO: 4; cathepsin B), LGLEGAD (SEQ ID NO: 5; cathepsin K), GPLD (SEQ ID NO: 6; subunit beta 1c), LGVLIV (SEQ ID NO: 7; cathepsin D), GLVLVA (SEQ ID NO: 8; cathepsin E), PAALVG (SEQ ID NO: 9; MMP2), GPAGLAG (SEQ ID NO: 10; MMP9), GGPLGVRGKK (SEQ ID NO: 11; MMP9), and GGfPRSGGGK (f=d-stereoisomer of phenylalanine; SEQ ID NO: 12; thrombin).

The enzyme-specific substrate may be optimized to provide both high catalytic activity (or other enzymatic activity) for specified target enzymes but to also release optimized detectable markers for detection. Patient outcome depends on the phenotype of individual diseases at the molecular level, and this is often reflected in expression of enzymes. The recent explosion of bioinformatics has facilitated exploration of complex patterns of gene expression in human tissues (Fodor, S. A. Massively parallel genomics. Science 277, 393-395 (1997)). Sophisticated computer algorithms have been recently developed capable of molecular diagnosis of tumors using the immense data sets generated by expression profiling (Khan J, Wei J S, Ringner M, Saal L H, Ladanyi M, Westermann F, et al. Classification and diagnostic prediction of cancers using gene expression profiling and artificial neural networks. Nat Med 2001; 7:673-679). This information can be accessed in order to identify enzymes and substrates associated with specific diseases. Based on this information the skilled artisan can identify appropriate enzyme or substrates to incorporate into the pro-diagnostic reagent.

Table 1 provides a non-limiting list of enzymes associated with (either increased or decreased with respect to normal) disease, the type of substrate, and in some instances, the specific substrate. Table 2 provides a non-limiting list of substrates associated with disease or other conditions. Numerous other enzyme/substrate combinations associated with specific diseases or conditions are known to the skilled artisan and are useful according to the invention.

TABLE 1
Non-limiting examples of cancer-associated enzymes and substrates.

| Disease | Enzyme | Substrate |
|---|---|---|
| Cancer | MMP | collagens, gelatin, |
| | | various ECM proteins |
| Cancer | MMP-2 | type IV collagen and |
| | | gelatin |
| Cancer | MMP-9 | type IV and V |
| | | collagens and gelatin |
| Cancer | Kallikreins | kininogens, |
| | | plasminogen |
| Cancer | Cathepsins | broad spectrum of |
| | | substrates |
| Cancer | plasminogen activator, tPA | Plasminogen |
| Cancer | Urokinase-type plasminogen | Plasminogen |
| | activator, uPA | |
| Cancer | ADAM (A Diseintegrin And | various extracellular |
| | Metalloprotease, also MDC, | domains of |
| | Adamalysin) | transmembrane |
| | | proteins |
| Pancreatic carcinoma | MMP-7 | various, e.g. collagen |
| | | 18, FasL, HLE, DCN, |
| | | IGFBP-3, MAG, |
| | | plasminogen, other |
| | | MMPs |
| Pancreatic Cancer | ADAM9, ADAM15 | various extracellular |
| | | domains of |
| | | transmembrane |
| | | proteins |
| Prostate adenocarcinoma | Matriptase, a type II | unspecific, cleaves |
| | transmembrane serine protease | after Lys or Arg |
| | | residues |
| Prostate cancer | Kallikrein 3 | kininogens, |
| | | plasminogen |
| Prostate cancer | ADAM15 | various extracellular |
| | | domains of |
| | | transmembrane |
| | | proteins |
| Ovarian carcinoma | Kallikrein 6 | kininogens, |
| | | plasminogen |
| Epithelial-derived tumors | Matriptase, a type II | unspecific, cleaves |
| (breast, prostate, ovarian, colon, | transmembrane serine protease | after Lys or Arg |
| oral) | | residues |
| Ovarian Cancer | MMP-2, MMP-9, kallikrein-10 | type IV and V |
| | (hk-10) | collagens and gelatin, |
| | | kininogens, |
| | | plasminogen |
| Breast, gastric, prostate cancer | cathepsins B, L and D | broad spectrum of |
| | | substrates |
| Endometrial cancer | cathepsin B | unspecific cleavage of |
| | | a broad spectrum of |
| | | substrates without |
| | | clear sequence |
| | | specificity |
| esophageal adenocarcinoma | cathepsin B | unspecific cleavage of |
| | | a broad spectrum of |
| | | substrates without |
| | | clear sequence |
| | | specificity |
| Invasive cancers, metastases | type II integral serine proteases | |
| | (dipeptidyl peptidase IV | |
| | (DPP4/CD26), seprase/fibroblast | |
| | activation protein alpha | |
| | (FAPalpha) and related type II | |
| | transmembrane prolyl serine | |
| | peptidases)) | |
| Invasive cancers, metastases | Seprase | various ECM proteins |
| Viral Infections | | |
| All Retroviruses | viral protease | precursor GagPol |
| | | fusion |
| HIV | HIV protease (HIV PR, an | precursor Gag and |
| | aspartic protease) | GagPol proteins |
| Hepatitis C | NS3 serine protease | viral precursor |
| | | polyprotein |
| Dengue | Dengue protease | autocleavage |
| | | (NS2B/NS3), |
| | | NS3/NS4A and |
| | | NS4B/NS5 cleavage |
| West Nile | NS2B/NS3pro | viral precursor |
| | | polyprotein |
| Bacterial Infections | | |
| Legionella spp. | zinc metalloprotease | Me-Arg-Pro-Tyr |
| Meninogencephalitis | histolytic cysteine protease | |
| Streptococcus pyogenes (Group | streptococcal pyrogenic exotoxin | extracellular matrix, |
| A Streptococcus) | B (SpeB) | immunoglobulins, |
| | | complement |
| | | components |
| Clostridium difficile | Cwp84 | fibronectin, laminin, |
| | | vitronectin and other |
| | | ECM proteins |
| Pseudomonas aeruginosa | lasA | Leu-Gly-Gly-Gly-Ala |
| Pseudomonas aeruginosa | Large ExoProtease A | Cleavage of peptide |
| | | ligands on PAR1, |
| | | PAR2, PAR4 |
| | | (Protease-activated |
| | | receptor). See, e.g., |
| | | Kida et al, Cell |
| | | Microbiol. 2008 |
| | | July; 10(7): 1491-504. |
| Pseudomonas aeruginosa | protease IV | complement factors, |
| | | fibrinogen, |
| | | plasminogen (See, e.g., |
| | | Engel et al., J Biol |
| | | Chem. 1998 July. |
| | | 3; 273(27): 16792-7). |
| Pseudomonas aeruginosa | alkaline protease | Complement factor |
| | | C2 (See, e.g., Laarman |
| | | et al., J Immunol. 2012 |
| | | January 1; 188(1): 386-93). |
| Additional Diseases | | |
| Alzheimer's disease | BACE-1,2 (Alzheimer secretase) | β-amyloid precursor |
| | | protein |
| Stroke and recovery | MMP, tPA | |
| cardiovascular disease | Angiotensin Converting Enzyme | angiotensin I, |
| | (ACE) | bradykinin |
| Atherosclerosis | cathepsin K, L, S | broad spectrum of |
| | | substrates |
| arthritis | MMP-1 | triple-helical fibrillar |
| | | collagens |
| rheumatoid arthritis | thrombin | Osteopontin |
| Malaria | SUB1 | KITAQDDEES |
| osteoarthritis | thrombin | Osteopontin |
| osteoporosis/osteoarthritis | cathepsin K, S | broad spectrum of |
| | | substrates |
| Arthritis, inflammatory joint | Aggrecanase (ADAMTS4, | aggrecans |
| disease | ADAMTS11) | (proteoglycans) |
| thrombosis | factor Xa (thrombokinase) | Prothrombin |
| thrombosis | ADAMTS13 | von Willebrand factor |
| | | (vWF) |
| thrombosis | plasminogen activator, tPA | Plasminogen |
| Stress-induced Renal pressure | Prostasin | epithelial Na channel |
| natriuresis | | subunits |

TABLE 2
Non-limiting examples of substrates associated with disease and other conditions.

| DISEASE | TARGET SUBSTRATE | ENZYME |
|---|---|---|
| Inflammation | Interleukin 1 beta | MMP-2, MMP-3, MMP-9, |
| | | Trypsin, chymotrypsin, pepsin, |
| | | Lys-C, Glu-C, Asp-N, Arg-C |
| Pituitary gland | IGFBP-3 | MMP-1, MMP-3, MMP-9, |
| dysfunction, abnormal | | Trypsin, chymotrypsin, pepsin, |
| bone density, growth | | Lys-C, Glu-C, Asp-N, Arg-C |
| disorders | | |
| Cancer | TGF-beta | MMP-9, Trypsin, chymotrypsin, |
| | | pepsin, Lys-C, Glu-C, Asp-N, |
| | | Arg-C |
| Cancer, autoimmune | TNF | MMP-7, Trypsin, chymotrypsin, |
| disease | | pepsin, Lys-C, Glu-C, Asp-N, |
| | | Arg-C |
| Cancer, autoimmune | FASL | MMP-7, Trypsin, chymotrypsin, |
| disease | | pepsin, Lys-C, Glu-C, Asp-N, |
| | | Arg-C |
| Wound healing, cardiac | HB-EGF | MMP-3, Trypsin, chymotrypsin, |
| disease | | pepsin, Lys-C, Glu-C, Asp-N, |
| | | Arg-C |
| Pfeiffer syndrome | FGFR1 | MMP-2, Trypsin, chymotrypsin, |
| | | pepsin, Lys-C, Glu-C, Asp-N, |
| | | Arg-C |
| Cancer | Decorin | MMP-2, MMP-3, MMP-7, |
| | | Trypsin, chymotrypsin, pepsin, |
| | | Lys-C, Glu-C, Asp-N, Arg-C |
| Cancer | Tumor associated | Endoglycosidases |
| | carbohydrate antigens | |
| Cancer | Sialyl Lewis^{a} | O-glycanase |
| Cancer | Sialyl Lewis^{X} | O-glycanase |
| Cancer/Rheumatoid | VEGF | Trypsin, chymotrypsin, pepsin, |
| Arthritis, pulmonary | | Lys-C, Glu-C, Asp-N, Arg-C |
| hypertension | | |
| Cancer | EGF | Trypsin, chymotrypsin, pepsin, |
| | | Lys-C, Glu-C, Asp-N, Arg-C |
| Cancer | IL2 | Trypsin, chymotrypsin, pepsin, |
| | | Lys-C, Glu-C, Asp-N, Arg-C |
| Cancer | IL6 | Trypsin, chymotrypsin, pepsin, |
| inflammation/angiogenesis | | Lys-C, Glu-C, Asp-N, Arg-C |
| Cancer | IFN-γ | Trypsin, chymotrypsin, pepsin, |
| | | Lys-C, Glu-C, Asp-N, Arg-C |
| Cancer | TNF-α | Trypsin, chymotrypsin, pepsin, |
| inflammation/angiogenesis, | | Lys-C, Glu-C, Asp-N, Arg-C |
| Rheumatoid Arthritis | | |
| Cancer, Pulmonary | TGF-β | Trypsin, chymotrypsin, pepsin, |
| fibrosis, Asthma | | Lys-C, Glu-C, Asp-N, Arg-C |
| Cancer, Pulmonary | PDGF | Trypsin, chymotrypsin, pepsin, |
| hypertension | | Lys-C, Glu-C, Asp-N, Arg-C |
| Cancer, pulmonary | Fibroblast growth factor | Trypsin, chymotrypsin, pepsin, |
| cystadenoma | (FGF) | Lys-C, Glu-C, Asp-N, Arg-C |
| Cancer | Brain-derived neurotrophic | Trypsin, chymotrypsin, pepsin, |
| | factor (BDNF) | Lys-C, Glu-C, Asp-N, Arg-C |
| Cancer | Interferon regulatory | Trypsin, chymotrypsin, pepsin, |
| | factors (IRF-1, IRF-2) | Lys-C, Glu-C, Asp-N, Arg-C |
| Inhibitor of tumor | MIF | Trypsin, chymotrypsin, pepsin, |
| suppressors | | Lys-C, Glu-C, Asp-N, Arg-C |
| Lymphomas/carcinomas, | GM-CSF | Trypsin, chymotrypsin, pepsin, |
| alveolar proteinosis | | Lys-C, Glu-C, Asp-N, Arg-C |
| Cancer invasion | M-CSF | Trypsin, chymotrypsin, pepsin, |
| | | Lys-C, Glu-C, Asp-N, Arg-C |
| Chemical carcinogenesis, | IL-12 | Trypsin, chymotrypsin, pepsin, |
| multiple sclerosis, | | Lys-C, Glu-C, Asp-N, Arg-C |
| rheumatoid arthritis, | | |
| Crohn's disease | | |
| Natural Killer T cell | IL-15 | Trypsin, chymotrypsin, pepsin, |
| leukemias, inflammatory | | Lys-C, Glu-C, Asp-N, Arg-C |
| bowel disease, rheumatoid | | |
| arthritis | | |
| Cirrhosis | Tissue inhibitor of MMPs | Trypsin, chymotrypsin, pepsin, |
| | (TIMPs) | Lys-C, Glu-C, Asp-N, Arg-C |
| Cirrhosis | Collagen I, III | MMP-1, MMP-8, Trypsin, |
| | | chymotrypsin, pepsin, Lys-C, |
| | | Glu-C, Asp-N, Arg-C |
| Cirrhosis | Collagen IV, V | MMP-2, Trypsin, chymotrypsin, |
| | | pepsin, Lys-C, Glu-C, Asp-N, |
| | | Arg-C |

Non-limiting examples of enzyme cleavable linkers may also be found in WO2010/101628, entitled METHODS AND PRODUCTS FOR IN VIVO ENZYME PROFILING, which was filed on Mar. 2, 2010.

In some embodiments, the enzyme-specific substrate is a cancer substrate. As used herein, “cancer substrate” refers to a substrate that is capable of being cleaved by a protease that is present (or upregulated) in a subject having a cancer (e.g., a malignant tumor, metastatic cancer, etc.). For example, certain cancers (e.g. metastatic cancers) are associated with upregulation of specific enzymes (e.g. ADAM28, MMP9, MMP12, ACE, C2, ADAMTS5, HTRA4, MMP16, MMP1, MMP3, MMP4, MMP7, MMP8, Cathepsin B, Cathepsin L, Cathepsin S, ADAM10, ADAM12, PRSS3, uPA, etc.). In some embodiments, the cancer substrate is a cancer metastasis substrate. In some embodiments, the cancer is colorectal cancer (e.g., CRC).

A substrate may be attached directly to the scaffold. For instance it may be coated directly on the surface of microparticles using known techniques, or chemically bonded to a polymeric scaffold, such as a PEG scaffold (e.g., via a peptide bond). Additionally, the substrate may be connected to the scaffold through the use of a linker. As used herein “linked” or “linkage” means two entities are bound to one another by any physicochemical means. Any linkage known to those of ordinary skill in the art, covalent or non-covalent, is embraced. Thus, in some embodiments the scaffold has a linker attached to an external surface, which can be used to link the substrate. Another molecule can also be attached to the linker. In some embodiments, two molecules are linked using a transpeptidase, for example, Sortase A.

The substrate is preferably a polymer made up of a plurality of chemical units. A “chemical unit” as used herein is a building block or monomer which may be linked directly or indirectly to other building blocks or monomers to form a polymer (e.g., a multi-arm PEG scaffold).

### Detectable Markers

A protease imaging sensor of the present disclosure comprises (i) a first detectable marker that is attached to an enzyme-specific substrate and (ii) a tumor imaging agent comprising a second detectable marker. Any of the detectable markers described herein may be capable of being released from the protease imaging when exposed to an enzyme (e.g., exposed to an in vivo or in vitro). In some embodiments, the first detectable marker is capable of being released from the sensor when exposed to an enzyme (e.g., exposed to an in vivo or in vitro). In some embodiments, at least one of the detectable markers on the protease imaging sensor is not capable of being released from the protease imaging sensor when exposed to an enzyme (e.g., exposed to an in vivo or in vitro). In some embodiments, the second detectable marker is not capable of being released from the protease imaging sensor when exposed to an enzyme (e.g., exposed to an in vivo or in vitro). In certain embodiments, a protease imaging sensor comprises (i) a scaffold linked to an enzyme-specific substrate that is attached to a first detectable marker and the first detectable marker is capable of being released from the sensor when exposed to an enzyme and (ii) a tumor imaging agent comprising a second detectable marker, the tumor imaging agent is linked to the scaffold, and the second detectable marker is not capable of being released from the protease imaging sensor when exposed to an enzyme. In some embodiments, the tumor imaging agent does not include a cell penetrating domain.

A detectable marker once released is free to travel to a remote site for detection. A remote site is used herein to refer to a site in the body that is distinct from the bodily tissue housing the enzyme where the enzymatic reaction occurs. In some embodiments, the bodily tissue housing the enzyme where the enzymatic reaction occurs is disease tissue (e.g., tumor tissue). For example, the bodily tissue housing the enzyme where the enzymatic reaction occurs may be a site of a primary cancer or may be a site of a metastasis. Sites of metastasis may vary with the type of cancer. Non-limiting sites of metastasis for various cancer types are provided in Table 3 (see, e.g., National Cancer Institute's description of metastatic cancer).

TABLE 3
Non-limiting sites of metastasis by cancer type.

| Cancer Type | Sites of Metastasis |
|---|---|
| Bladder | Bone, liver, lung |
| Breast | Bone, brain, liver, lung |
| Colon | Liver, lung, peritoneum |
| Kidney | Adrenal gland, bone, brain, liver, lung |
| Lung | Adrenal gland, bone, brain, liver, other lung |
| Melanoma | Bone, brain, liver, lung, skin, muscle |
| Ovary | Liver, lung, peritoneum |
| Pancreas | Liver, lung, peritoneum |
| Prostate | Adrenal gland, bone, liver, lung |
| Rectal | Liver, lung, peritoneum |
| Stomach | Liver, lung, peritoneum |
| Thyroid | Bone, liver, lung |
| Uterus | Bone, liver, lung, peritoneum, vagina |

Modification of the protease-specific substrate by an enzyme in vivo, results in the production of a first detectable marker (e.g., release or decoupling of the detectable marker from the scaffold upon cleavage of the protease-specific substrate by an enzyme). Any of the detectable markers described herein is a detectable molecule. A detectable marker can be part of the substrate, e.g. the piece that is released or added upon cleavage or it can be a separate entity. In some embodiments, a detectable marker is composed of two ligands joined by a linker (e.g., a fluorescence resonance energy transfer (FRET) pair). A detectable marker may be comprised of, for instance one or more of a peptide, nucleic acid, small molecule, fluorophore/quencher, carbohydrate, particle, radiolabel, MRI-active compound, inorganic material, or organic material, with encoded characteristics to facilitate optimal detection, or any combination thereof. In some embodiments, the detectable marker comprises a GluFib peptide (SEQ ID NO: 13; EGVNDNEEGFFSAR) conjugated to a capture ligand and/or a fluorophore (e.g., a GluFib peptide flanked by a capture ligand, such as biotin, and a fluorophore, such as FAM).

In some embodiments, a substrate comprises a capture ligand, which is a molecule that is capable of being captured by a binding partner. The detection ligand is a molecule that is capable of being detected by any of a variety of methods. While the capture ligand and the detection ligand will be distinct from one another in a particular detectable marker, the class of molecules that make us capture and detection ligands overlap significantly. For instance, many molecules are capable of being captured and detected. In some instances these molecules may be detected by being captured or capturing a probe. The capture and detection ligand each independently may be one or more of the following: a protein, a peptide, a polysaccharide, a nucleic acid, a fluorescent molecule, or a small molecule, for example. In some embodiments the detection ligand or the capture ligand may be, but is not limited to, one of the following: Alexa488, TAMRA, DNP, fluorescein, OREGON GREEN® (4-(2,7-difluoro-6-hydroxy-3-oxo-3H-xanthen-9-YL)isophthalic acid), TEXAS RED® (sulforhodamine 101 acid chloride), Dansyl, BODIPY® (boron-dipyrromethene), Alexa405, CASCADE BLUE® (Acetic acid, [(3,6,8-trisulfo-1-pyrenyl)oxy]-, 1-hydrazide, trisodium salt), Lucifer Yellow, Nitrotyrosine, HA-tag, FLAG-tag, His-tag, Myc-tag, V5-tag, S-tag, biotin or streptavidin.

In some embodiments, the capture ligand and a detection ligand are connected by a linker. The purpose of the linker is prevent steric hindrance between the two ligands. Thus, the linker may be any type of molecule that achieves this. The linker may be, for instance, a polymer such as PEG, a protein, a peptide, a polysaccharide, a nucleic acid, or a small molecule. In some embodiments the linker is a protein of 10-100 amino acids in length. In other embodiments the linker is GluFib (SEQ ID NO: 13; EGVNDNEEGFFSAR). Optionally, the linker may be 8 nm-100 nm, 6 nm-100 nm, 8 nm-80 nm, 10 nm-100 nm, 13 nm-100 nm, 15 nm-50 nm, or 10 nm-50 nm in length.

In some embodiments, a detectable marker is a ligand encoded reporter. Without wishing to be bound by any particular theory, a ligand encoded reporter binds to a target molecule (e.g., a target molecule present in a tumor), allowing for detection of the target molecule at a site remote from where the ligand encoded reporter bound to the target (e.g., at a sight remote from a tumor).

In some embodiments, a detectable marker is a mass encoded reporter, for example an iCORE as described in WO2012/125808, filed Mar. 15, 2012, the entire contents of which are incorporated herein by reference. Upon arrival in the diseased microenvironment, the iCORE agents interface with aberrantly active proteases to direct the cleavage and release of surface-conjugated, mass-encoded peptide substrates into host urine for detection by mass spectrometry (MS) as synthetic biomarkers of disease.

A detectable marker may be detected by any known detection methods to achieve the capture/detection step. A variety of methods may be used, depending on the nature of the detectable marker. Detectable markers may be directly detected, following capture, through optical density, radioactive emissions, non-radiative energy transfers, or detectable markers may be indirectly detected with antibody conjugates, affinity columns, streptavidin-biotin conjugates, PCR analysis, DNA microarray, optical imaging, magnetic resonance (MR) imaging, positron emission tomography (PET) imaging, intraoperative imaging, and fluorescence analysis.

A capture assay, in some embodiments, involves a detection step selected from the group consisting of an ELISA, including fluorescent, colorimetric, bioluminescent and chemiluminescent ELISAs, a paper test strip or lateral flow assay (LFA), bead-based fluorescent assay, and label-free detection, such as surface plasmon resonance (SPR). The capture assay may involve, for instance, binding of the capture ligand to an affinity agent.

The analysis (e.g., detecting) step may be performed directly on a biological sample (e.g., urine sample, blood sample, tissue sample, etc.) or the signature component may be purified to some degree first. For instance, a purification step may involve isolating the detectable marker from other components in a biological sample (e.g., urine sample, blood sample, tissue sample, etc.). Purification steps include methods such as affinity chromatography. As used herein an “isolated molecule” or “purified molecule” is a detectable marker that is isolated to some extent from its natural environment. The isolated or purified molecule need not be 100% pure or even substantially pure prior to analysis.

The methods for analyzing detectable markers by identifying the presence of a detectable marker may be used to provide a qualitative assessment of the molecule (e.g., whether the detectable marker is present or absent) or a quantitative assessment (e.g., the amount of detectable marker present to indicate a comparative activity level of the enzymes). The quantitative value may be calculated by any means, such as, by determining the percent relative amount of each fraction present in the sample. Methods for making these types of calculations are known in the art.

A detectable marker described herein may be labeled. For example, a label may be added directly to a nucleic acid when the isolated detectable marker is subjected to PCR. For instance, a PCR reaction performed using labeled primers or labeled nucleotides will produce a labeled product. Labeled nucleotides (e.g., fluorescein-labeled CTP) are commercially available. Methods for attaching labels to nucleic acids are well known to those of ordinary skill in the art and, in addition to the PCR method, include, for example, nick translation and end-labeling.

Labels suitable for use in the methods of the present invention include any type of label detectable by standard means, including spectroscopic, photochemical, biochemical, electrical, optical, or chemical methods. Preferred types of labels include fluorescent labels such as fluorescein. A fluorescent label is a compound comprising at least one fluorophore. Commercially available fluorescent labels include, for example, fluorescein phosphoramidides such as fluoreprime (Pharmacia, Piscataway, NJ), fluoredite (Millipore, Bedford, MA), FAM (ABI, Foster City, CA), rhodamine, polymethadine dye derivative, phosphores, Texas red, green fluorescent protein, CY3, and CY5. Polynucleotides can be labeled with one or more spectrally distinct fluorescent labels. “Spectrally distinct” fluorescent labels are labels which can be distinguished from one another based on one or more of their characteristic absorption spectra, emission spectra, fluorescent lifetimes, or the like. Spectrally distinct fluorescent labels have the advantage that they may be used in combination (e.g., “multiplexed”). Radionuclides such as ³H, ¹²⁵I, ³⁵S, ¹⁴C, or ³²P are also useful labels according to the methods of the invention. A plurality of radioactively distinguishable radionuclides can be used. Such radionuclides can be distinguished, for example, based on the type of radiation (e.g. α, β, or δ radiation) emitted by the radionuclides. The ³²P signal can be detected using a phosphoimager, which currently has a resolution of approximately 50 microns. Other known techniques, such as chemiluminescence or colormetric (enzymatic color reaction), can also be used.

Detectable markers also include diagnostic and imaging labels (e.g., radiolabels). For example, a detectable marker may be detectable by positron emission tomography or computerized tomography. Non-limiting examples of detectable markers include detectable markers that are suitable for in vivo imaging, which include magnetic resonance imaging (MRI): Gd(DOTA); for nuclear medicine: ²⁰¹Tl, gamma-emitting radionuclide ^{99m}Tc; for positron-emission tomography (PET): positron-emitting isotopes, ¹⁸F-2-deoxyfluoroglucose (FDG), (18)F-fluoride, copper-64 (⁶⁴Cu), gadodiamide, and radioisotopes of Pb(II) such as ²⁰³Pb; ¹¹¹In; and ⁸⁹Zr.

In some embodiments, a detectable marker comprises a metal chelator (e.g., 1,4,7-Triazacyclononane-1,4,7-triacetic acid (NOTA), 1,4,7,10-tetraazacyclododecane-N,N′,N″,N′″-tetraacetic acid (DOTA), Diethylenetriaminepentaacetic Anhydride (DTPA), 1,4,8,11-tetraazacyclotetradecane-1,4,8,11-tetraacetic acid (TETA), and/or deferoxamine (e.g., for ⁸⁹Zr).

Quencher compositions in which a “donor” fluorophore is joined to an “acceptor” chromophore by a short bridge that is the binding site for the enzyme may also be used. The signal of the donor fluorophore is quenched by the acceptor chromophore through a process believed to involve resonance energy transfer (RET). Cleavage of the peptide results in separation of the chromophore and fluorophore, removal of the quench, and generation of a subsequent signal measured from the donor fluorophore.

### Tumor Imaging Agent

The tumor imaging agent (e.g., a tumor imaging agent) of the present disclosure comprise a detectable marker and allow for localization of tumor cells. The tumor imaging agents may be capable of associating (e.g., binding) with a target tissue.

In some embodiments, the tumor imaging agent does not include a cell penetrating domain. As used herein, a cell penetrating domain is an agent (e.g., peptide sequence or a non-peptide) that promotes cellular uptake of itself and any conjugated cargo. Non-limiting examples of cell penetrating domains include protein transduction domains (e.g., TAT and penetratin), primary amphipathic cell penetrating peptides, secondary amphipathic cell penetrating peptides, and nonamphipathic cell penetrating peptides. See, e.g., Madani et al., J Biophys. 2011; 2011: 414729.

In some embodiments, the tumor imaging agent comprises a tumor insertion agent, which allows for localization of the tumor imaging agent to a tumor (e.g., a tumor vivo). Non-limiting examples of tumor insertion agents include pH low insertion peptides. pH low insertion peptides are capable of inserting across a lipid bilayer at a low or acidic pH, but do not insert into a lipid bilayer at neutral or basic pH. In some embodiments, a low pH is a pH that is a pH value that is less than 7, less than 6, less than 5, less than 4, less than 3, less than 2 or less than 1. In some embodiments, a neutral pH is a pH value of about 7. In some embodiments, a basic pH is a pH value that is higher than 7, higher than 8, higher than 9, higher than 10, higher than 11, higher than 12, higher than 13 or higher than 14.

The tumor imaging agents of the present disclosure may comprise an amino acid sequence set forth in Table 4 and/or Table 5. In some embodiments, a tumor imaging agent comprises a sequence that is at least 70% (e.g., at least 75%, 80%, 85%, 90%, 95%, 98%, 99%, or 100%) identical to a sequence set forth in Table 4 and/or Table 5.

In some embodiments, a pH low insertion peptide may comprise D-form amino acids, an azide side chain (N3), and/or cyanine (Cy7).

In certain embodiments, a pH low insertion peptide may comprise the amino acid sequence WKK. In certain embodiments, the tryptophan in the amino acid sequence WKK is a D-amino acid. In certain embodiments, all amino acids in the WKK sequence are D-amino acids. In certain embodiments, all amino acids in the WKK sequence are L-amino acids. In certain embodiments, the lysines in the WKK sequence are D-amino acids. In certain embodiments, the tryptophan in the amino acid sequence WKK is a L-amino acid, while the lysines are D-amino acids.

TABLE 4
Non-limiting examples of pH low insertion peptide
sequences.

| | SEQ ID |
|---|---|
| Sequence | NO: |
| ACEQNPIYWARYADWLFTTPLLLLDLALLVDADEGT | 14 |
| ACEDQNPYWARYADWLFTTPLLLLDLALLVDG | 15 |
| ACEDQNPYWRAYADLFTPLTLLDLLALWDG | 16 |
| ACDDQNPWRAYLDLLFPTDTLLLDLLW | 17 |
| ACEEQNPWRAYLELLFPTETLLLELLW | 18 |
| ACDDQNPWARYLDWLFPTDTLLLDL | 19 |
| CDNNNPWRAYLDLLFPTDTLLLDW | 20 |
| ACEEQNPWARYLEWLFPTETLLLEL | 21 |
| CEEQQPWAQYLELLFPTETLLLEW | 22 |
| CEEQQPWRAYLELLFPTETLLLEW | 23 |
| ACEDQNPWARYADWLFPTTLLLLD | 24 |
| ACEEQNPWARYAEWLFPTTLLLLE | 25 |
| ACEDQNPWARYADLLFPTTLAW | 26 |
| ACEEQNPWARYAELLFPTTLAW | 27 |
| Ac-TEDADVLLALDLLLLPTTFLWDAYRAWYPNQECA-Am | 28 |
| CDDDDDNPNYWARYANWLFTTPLLLLNGALLVEAEET | 29 |
| CDDDDDNPNYWARYAPWLFTTPLLLLPGALLVEAEET | 30 |

A tumor imaging agent may comprise a tumor targeting agent may be conjugated (e.g., N-terminally conjugated or C-terminally conjugated) to a detectable marker (e.g., including a fluorophore, radiolabel, or any combination thereof). In some embodiments, a tumor imaging agent comprises a detectable marker and a tumor insertion agent (e.g., a pH low insertion peptide). The tumor insertion agent may comprises two ends (e.g., a N-terminus and a C-terminus). In certain embodiments, the tumor insertion agent is conjugated to a detectable marker (e.g., a fluorophore or a radiolabel) on one end and is conjugated to a scaffold (e.g., a scaffold comprising polyethylene glycol) on the other end. Non-limiting methods of conjugation include click chemistry. See, e.g., Thirumurugan et al., Chem Rev. 2013 Jul. 10; 113(7):4905-79.

As used herein, “conjugated” means two entities stably bound to one another by any physiochemical means. It is important that the nature of the attachment is such that it does not impair substantially the effectiveness of either entity. Keeping these parameters in mind, any covalent or non-covalent linkage known to those of ordinary skill in the art may be employed. In some embodiments, covalent linkage is preferred. Noncovalent conjugation includes hydrophobic interactions, ionic interactions, high affinity interactions such as biotin avidin and biotin streptavidin complexation and other affinity interactions. Such means and methods of attachment are well known to those of ordinary skill in the art.

### Methods to Detect Enzyme Activity

Aspects of the disclosure relate to the surprising discovery that sensors comprising a tumor imaging agent and an enzyme-specific substrate attached to a detectable marker are useful for detecting enzyme activity (e.g., in vitro and in vivo).

In some embodiments, detection of a detectable marker that has been released from the sensor in a biological sample (e.g., in vitro or in vivo) is indicative of enzyme (e.g., cancer-associated enzyme) activity. In some embodiments, detection of a detectable marker that is part of the tumor imaging agent indicates the site of exposure to the enzyme (e.g., cancer-associated enzyme).

As used herein, a biological sample is a tissue sample (such as a blood sample, a hard tissue sample, a soft tissue sample, etc.), a urine sample, mucous sample, saliva sample, fecal sample, seminal fluid sample, cerebrospinal fluid sample, etc. In preferred embodiments, the biological sample is a tissue sample. The tissue sample may be obtained from any tissue of the subject, including brain, lymph node, breast, liver, pancreas, colon, liver, lung, blood, skin, ovary, prostate, kidney, or bladder. The tissue from which the biological sample is obtained may be healthy or diseased. In some embodiments, a tissue sample comprises tumor cells or a tumor.

A tissue sample for use in methods described by the disclosure may be unmodified (e.g., not treated with any fixative, preservative, cross-linking agent, etc.) or physically or chemically modified. Examples of fixatives include aldehydes (e.g., formaldehyde, formalin, gluteraldehyde, etc.), alcohols (e.g., ethanol, methanol, acetone, etc.), and oxidizing agents (e.g., osmium tetroxide, potassium dichromate, chromic acid, potassium permanganate, etc.). In some embodiments, a tissue sample is cryopreserved (e.g., frozen). In some embodiments, a tissue sample is embedded in paraffin.

### Methods for Detecting a Tumor in a Subject

In some aspects, the disclosure provides methods for a tumor in a subject. The subject may be suspected of having a tumor, at risk for having a tumor, or has a tumor. As used herein, a subject is a human, non-human primate, cow, horse, pig, sheep, goat, dog, cat, or rodent. In all embodiments human subjects are preferred. In aspects of the invention pertaining to disease diagnosis in general the subject preferably is a human suspected of having a disease, or a human having been previously diagnosed as having a disease. Methods for identifying subjects suspected of having a disease may include physical examination, subject's family medical history, subject's medical history, biopsy, or a number of imaging technologies such as ultrasonography, computed tomography, magnetic resonance imaging, magnetic resonance spectroscopy, or positron emission tomography.

In some embodiments, methods described by the disclosure result in identification (e.g., detection) of a disease in a subject prior to the onset of symptoms. In some embodiments, a tumor that is less than 1 cm, less than 0.5 cm, or less than 0.005 cm is detected using methods described by the disclosure. In some embodiments, the tumor that is detected is between 1 mm and 5 mm in diameter (e.g., about 1 mm, 2 mm, 3 mm, 4 mm, or about 5 mm) in diameter.

In some embodiments, the presence of enzyme activity (e.g., protease activity) in a subject is identified by obtaining a biological sample from a subject that has been administered a sensor as described by the disclosure and detecting the presence of a detectable marker in the biological sample. Generally, the biological sample may be a tissue sample (such as a blood sample, a hard tissue sample, a soft tissue sample, etc.), a urine sample, saliva sample, fecal sample, seminal fluid sample, cerebrospinal fluid sample, etc. In some embodiments, detection of a detectable marker (e.g., a detectable marker that was released from a sensor) is indicative of the subject having a tumor.

In some embodiments, the site of exposure to an enzyme (e.g., cancer-associated enzyme) is identified by detecting the detectable marker from the tumor imaging agent in a subject that has been administered a sensor as described by the present disclosure.

In some embodiments, the methods described herein comprise detecting at least one detectable marker. In some embodiments, the detectable marker that has been released from an enzyme-specific substrate is detected. In some embodiments, the detectable marker that is part of the tumor imaging agent is detected. In some embodiments, at least two detectable markers are detected. For example, a detectable marker may be detected in a biological sample (e.g., a detectable marker that has been released from a sensor) and a detectable marker may be detected in situ in a subject that has been administered the sensor. Any suitable method may be used to detect any of the detectable markers described herein, including fluorescence analysis, optical imaging, magnetic resonance imaging (MRI), positron emission tomography (PET) imaging, computerized tomography (CT) imaging, intraoperative imaging, or any combination thereof.

Detection of one or more detectable markers in the biological sample may be indicative of a subject having a cancer (e.g., colorectal cancer). In some instances, detection of one or more detectable markers in the biological sample is indicative of a specific stage of a disease (e.g., metastatic or non-metastatic). In some embodiments, detection of one or more detectable markers in the biological sample is indicative of a type of cancer.

Detection of one or more detectable markers in vivo in a subject (e.g., detection of a tumor imaging agent) may be indicative of whether a subject has metastatic or non-metastatic cancer.

Detection of one or more detectable markers in a biological sample from a subject administered a sensor of the present disclosure, in situ in a subject administered a sensor of the present disclosure, or any combination thereof may be used to monitor tumor progression in the subject. To practice this embodiment, one or more detectable markers is detected more than one time (e.g., at least 2, 3, 4, 5, 6, 7, 8, 9, 10, 15, 20, 30, 40, 50, 60, 70, 80, 90, or at least 100 times). In certain embodiments, the subject has been administered a first therapeutic agent, a first therapeutic intervention has been performed on the subject, or a combination thereof.

The methods of the present disclosure may further comprise continuing or modifying a subject's course of treatment (e.g., administering a second therapeutic agent, administering a different therapeutic agent, administering a first therapeutic agent, performing a therapeutic intervention, or stopping treatment) after detection one or more detectable markers. In some embodiments, lack of detection of a detectable marker over time is indicative of a subject no longer having a cancer (e.g., type of cancer or stage of cancer) and treatment may be modified or discontinued.

In certain embodiments, an increase the amount of a detectable marker (e.g., increase by at least 5%, 10%, 20%, 30%, 40%, 50%, 60%, 70%, 80%, 90%, 100%, 200%, 300%, 400%, 500%, 600%, 700%, 800%, 900%, or 1000%) detected in a subsequent biological sample (e.g., a tissue sample (such as a blood sample, a hard tissue sample, a soft tissue sample, etc.), a urine sample, mucous sample, saliva sample, fecal sample, seminal fluid sample, or cerebrospinal fluid sample) from a subject administered a sensor of the present disclosure compared to one or more previous samples from the subject indicates that a tumor is progressing. In certain embodiments, a similar amount of a detectable marker (e.g., no change in the amount, or a change (increase or decrease) of 1-5%) detected in a subsequent biological sample (e.g., a tissue sample (such as a blood sample, a hard tissue sample, a soft tissue sample, etc.), a urine sample, mucous sample, saliva sample, fecal sample, seminal fluid sample, or cerebrospinal fluid sample) from a subject administered a sensor of the present disclosure compared to one or more previous samples from the subject indicates that a tumor is stable. In certain embodiments, a decrease the amount of a detectable marker (e.g., increase by at least 5%, 10%, 20%, 30%, 40%, 50%, 60%, 70%, 80%, 90%, 100%, 200%, 300%, 400%, 500%, 600%, 700%, 800%, 900%, or 1000%) detected in a subsequent biological sample (e.g., a tissue sample (such as a blood sample, a hard tissue sample, a soft tissue sample, etc.), a urine sample, mucous sample, saliva sample, fecal sample, seminal fluid sample, or cerebrospinal fluid sample) from a subject administered a sensor of the present disclosure compared to one or more previous samples from the subject indicates that a tumor is in remission.

Without being bound by a particular theory, a protease imaging sensor comprising a tumor imaging peptide with a detectable marker (e.g., a tracer) may produce a low signal when not localized to a cell (e.g., in the absence of a tumor), but produce a high signal that is detectable when localized to a cell (e.g., in the presence of a tumor).

### Administration

Compositions comprising any of the sensors described herein can be administered to any suitable subject. In some embodiments, the sensors of the disclosure are administered to the subject in an effective amount for detecting a tumor (e.g., a primary tumor, a metastatic tumor, or a combination thereof). An “effective amount”, for instance, is an amount necessary or sufficient to cause release of a detectable marker in the presence of an enzyme, detection of a tumor in situ, or a combination thereof. The effective amount of an sensor of the present disclosure described herein may vary depending upon the specific compound used, the mode of delivery of the compound, and whether it is used alone or in combination. The effective amount for any particular application can also vary depending on such factors as the disease being assessed or treated, the particular compound being administered, the size of the subject, or the severity of the disease or condition as well as the detection method. One of ordinary skill in the art can empirically determine the effective amount of a particular molecule of the invention without necessitating undue experimentation. Combined with the teachings provided herein, by choosing among the various active compounds and weighing factors such as potency, relative bioavailability, patient body weight, severity of adverse side-effects and preferred mode of administration, an effective regimen can be planned.

Pharmaceutical compositions of the present invention comprise an effective amount of one or more agents, dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences (1990), incorporated herein by reference). Except insofar as any conventional carrier is incompatible with the active ingredient, its use in the therapeutic or pharmaceutical compositions is contemplated. The agent may comprise different types of carriers depending on whether it is to be administered in solid, liquid or aerosol form, and whether it need to be sterile for such routes of administration as injection.

Aspects of the disclosure relate to systemic administration of an sensor to a subject. In some embodiments, the systemic administration is injection, optionally subcutaneous injection. The sensors of the present disclosure may also be administered through any suitable routes. For instance, the compounds of the present invention can be administered intravenously, intradermally, intratracheally, intraarterially, intralesionally, intratumorally, intracranially, intraarticularly, intraprostaticaly, intrapleurally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, injection, infusion, continuous infusion, localized perfusion bathing target cells directly, via a catheter, via a lavage, in creams, in lipid compositions (e.g., liposomes), or by other method or any combination of the forgoing as would be known to one of ordinary skill in the art (see, for example, Remington's Pharmaceutical Sciences (1990), incorporated herein by reference).

### EXAMPLES

### Protease-Responsive Imaging Sensors Detect and Image Colorectal Cancer (CRC) Metastasis.

This Example evaluated the ability of protease-responsive imaging sensors (PRISMs) to detect and image colorectal cancer (CRC) metastasis. In particular, mouse models of CRC liver and lung metastasis were used.

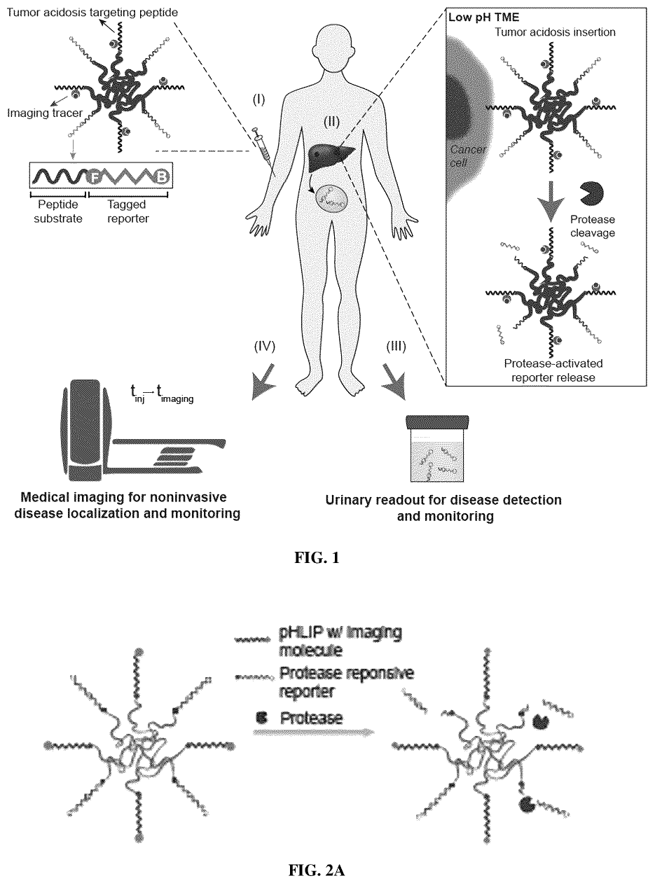

To advance sensitive detection of cancer metastases and increase efficacy of treatments, new sensors that harness metastasis-specific proteases as triggers to release urinary biomarkers for sensitive detection and imaging of tumor metastases were developed. This approach integrates the activatable release of a urinary reporter for early disease detection with a pathological pH-triggered signal amplification for the precise imaging of CRC metastases by PET/CT. Two strategies endowed ultrasensitivity to these sensors: (1) a peptide substrate susceptible to cleavage by a metastasis-specific protease sheds labeled fragments into the urine via proteolytic cleavage, and (2) a pH low insertion peptide (pHLIP) localizes the cancer-specific signal by engaging active tumor trafficking and insertion triggered by acidic microenvironments (FIG. 1, I & II).

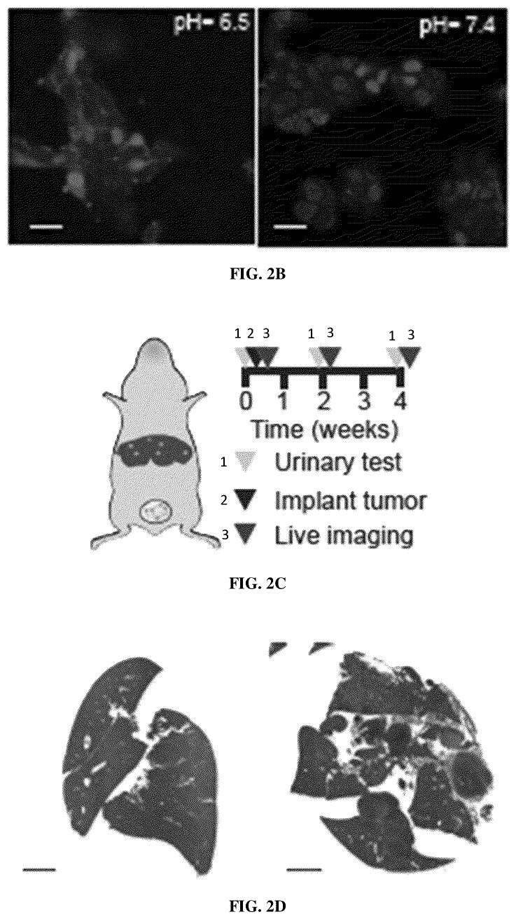

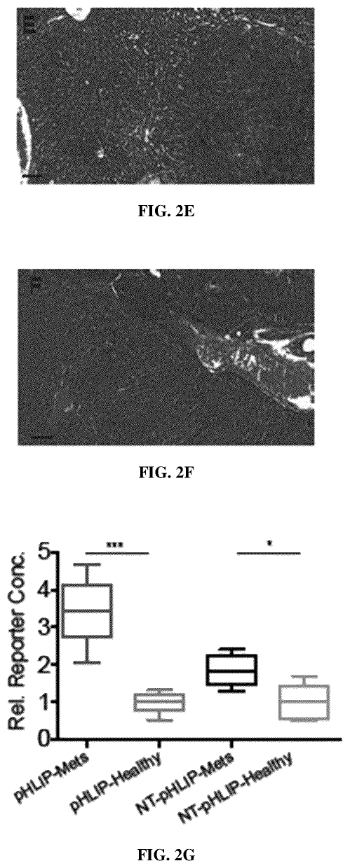

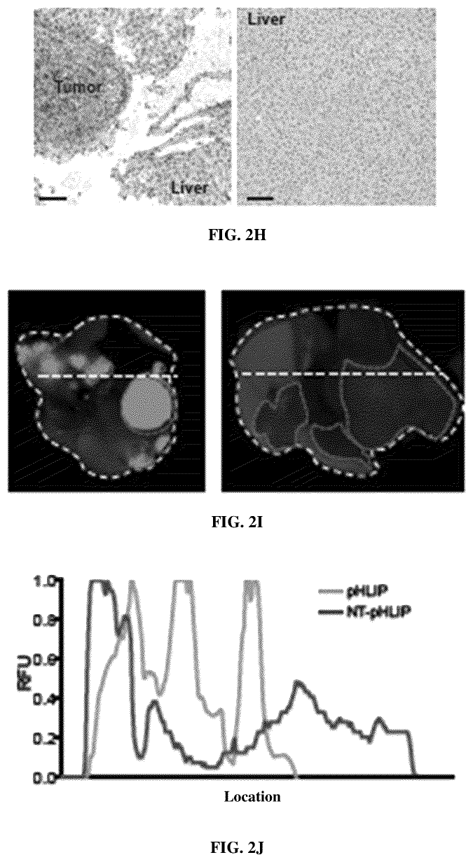

Activity-based urinary detection encompasses two steps of amplification by allowing single proteases to cleave thousands of substrates, which then are concentrated over 20 fold in the urine (from ˜5 L of blood to ˜0.2 L void volume). When applied to a disseminated ovarian cancer model, targeted activity-based sensors detected tumor nodules with median diameters of <2 mm, whereas the blood biomarker HE4 was only able to detect tumors at an average total burden of 88 mm³ (Kwon et al., Nat Biomed Eng. 2017; 1). With this level of predictive power, once the urinary signal turns positive, one can leverage advanced medical imaging to carefully screen and stratify patients in order to establish an effective treatment regimen (FIG. 1, III & IV). In addition to the powerful urinary signal detection, the advanced medical imaging, here PET/CT, provides a noninvasive approach that monitors disease progression in primary or metastatic tumors, and also patients' response to specific treatments (Longo et al., Cancer Res. 2016 Nov. 15; 76(22):6463-6470; Sun et al., Sci Transl Med. 2018 Mar. 7; 10(431); Farwell et al., Cancer. 2014 Nov. 15; 120(22):3433-45; Zinnhardt et al., Cancer Res. 2017 Apr. 15; 77(8):1831-1841). PET and CT are both standard imaging tools used by healthcare providers to pinpoint disease sites in the body. The PET scan demonstrates biological function of the body (i.e., cell physiology). The CT scan provides information on anatomy including size, shape, and location of structures in the body (Farwell et al., Cancer. 2014 Nov. 15; 120(22):3433-45). Combining these two noninvasive technologies, PET/CT can more accurately and quickly diagnose, stage, and monitor treatment for cancer. Radioactive PET tracers used in clinics, such as ¹⁸F-2-deoxyfluoroglucose (FDG), visualize differences in metabolic activity that accompany inflammation but lack the discriminatory power that tumor-targeting moieties (peptides, antibodies, or their fragments) might afford (Sun et al., Sci Transl Med. 2018 Mar. 7; 10(431); Rashidian et al., Proc Natl Acad Sci USA. 2015 May 12; 112(19):6146-51). However, these moieties tend to accumulate in kidneys and other organs of elimination, resulting in suboptimal signal-to-noise ratios. Considering the benefits of PEG-based reporters of protease cleavage and PEGylation for optimized imaging signal, a first iteration of PRISM was constructed using multivalent polyethylene glycol scaffolds. These sensors carried two functional components: (1) a peptide substrate that sheds labeled fragments into the urine via proteolytic cleavage of MMP9, which is significantly upregulated in both primary and metastatic CRC (Kwon et al., Nat Biomed Eng. 2017; 1), and (2) a pH low insertion peptide (pHLIP) that engages active tumor trafficking and insertion triggered by acidic microenvironments to localize the cancer-specific imaging signal (FIG. 2A). Specifically, both pHLIP and the MMP9-activated urinary reporter were immobilized on polychain PEG by click chemistry. By modifying pHLIP with fluorophores, tracers, or a combination thereof at the N-terminus, one can enable tumor-specific imaging with desired imaging techniques. The scaffold (8-arm PEG) was directly conjugated with the enzyme-specific substrate and the pH low insertion peptide through covalent linkage, using DBCO-azide click chemistry.

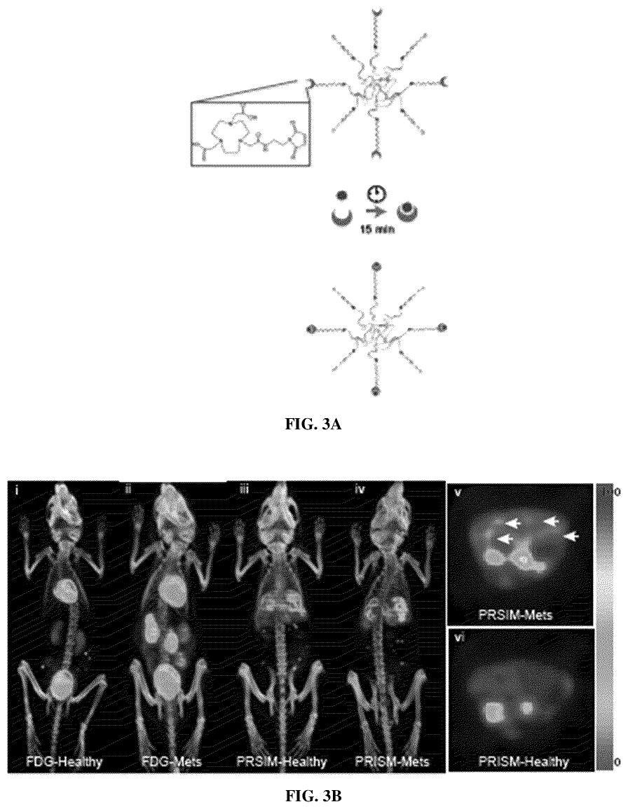

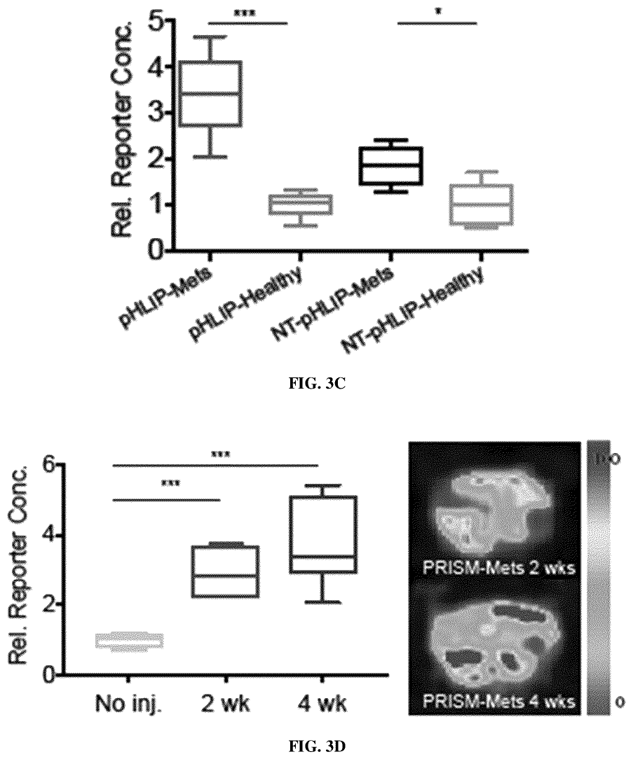

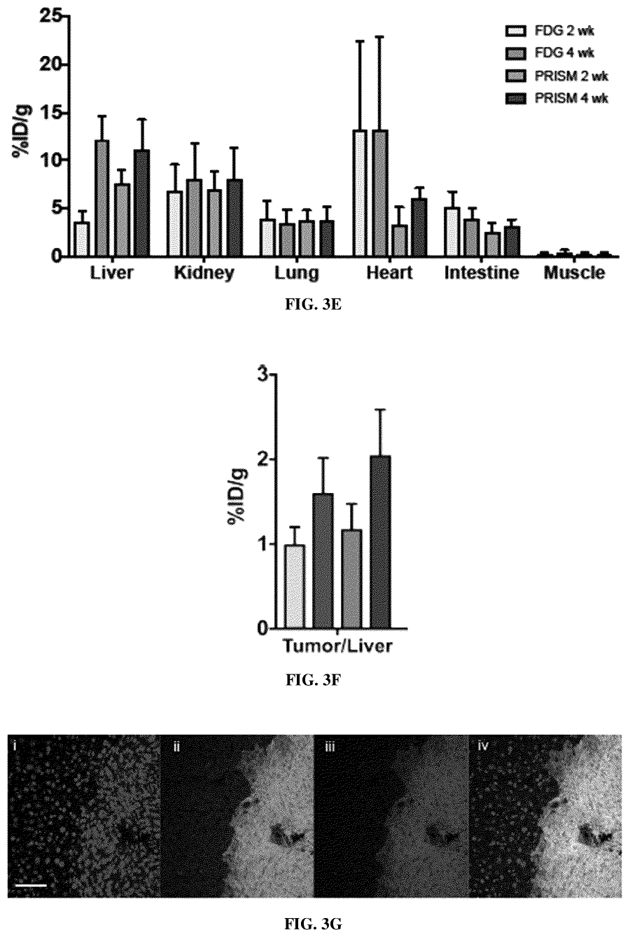

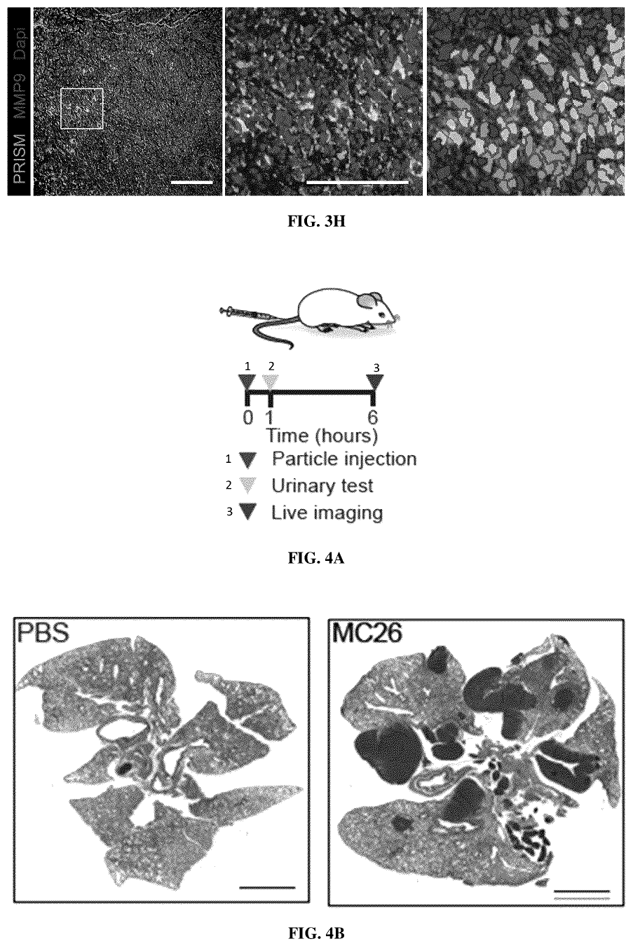

PRISM is 15 nm in diameter and carries ˜1:1 of pHLIP and MMP9-activated urinary reporters. When incubated with cancer cells at acidic pH (6.5), PRISM exhibited significantly higher cell accumulation than that observed at pH 7.4 (FIG. 2B). An intrasplenic injection model of liver metastases was developed. Following a midline incision and externalization of the spleen in Balb/c mice, the luciferized MMP9-secreting CRC cell line (MC26) was inoculated into the subsplenic capsule, allowed cells to traverse vasculature to seed the liver, and then removed the spleen to prevent ectopic tumor growth (FIGS. 2C, 2D, 2E & 2F) (Danino et al., Sci Transl Med. 2015 May 27; 7(289):289ra84). Upon intravenous administration of sensors, urine samples from tumor-bearing and healthy control mice were collected after 1 hr, and the reporter levels were detected by ELISA. Comparison of urinary signals generated by two sets of sensors bearing pHLIP or its non-targeting counterpart revealed a 1.8-fold increase in cancer-specific signal generation, suggesting pH-selective targeting of PRISM enhanced sensitivity of detection (FIG. 2G). At 2 and 6 hr post-injection, mice underwent small animal PET/CT imaging for longitudinal assessment of pHLIP reporter. 1,4,7-triazacyclononane-triacetic acid (NOTA) was site-specifically conjugated to pHLIP by maleimide chemistry (FIG. 3A). For real-time in vivo imaging, singly-labeled ⁶⁴Cu successfully imaged CRC liver and lung metastases in immunocompetent Balb/c mice, resulting in positive-to-negative tumor ratios of 2.2:1 (FIG. 3B). Quantification of urinary reporters collected at different time points, and longitudinal observation of individual animals indicated growth of metastatic sites in the liver (FIG. 3D). The PRISM probe was benchmarked against the conventional PET tracer used in clinics, ¹⁸F-FDG, and found that the standard uptake value in major organs such as liver, kidney, lung, intestine are comparable (FIG. 3E). In particular, similar tumor versus liver ratios were observed in mice imaged with ¹⁸F-FDG and ⁶⁴Cu-PRISM, respectively, demonstrating that PRISM is effective in imaging of metastases in vivo (FIG. 3F). At the end of the determined time point, the mice were sacrificed and livers with metastases were collected and subjected to ex vivo immunofluorescence and immunohistochemistry analysis. The extracellular protease, MMP9, accumulated along the metastasis invasion front, and PRISM sensors were colocalized with abnormal physiologic conditions such as hypoxia (FIGS. 2H and 3G).

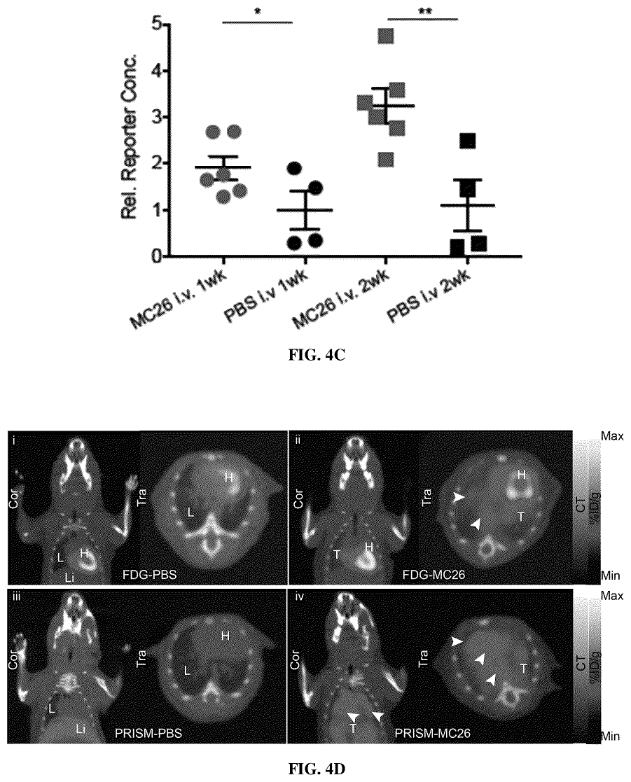

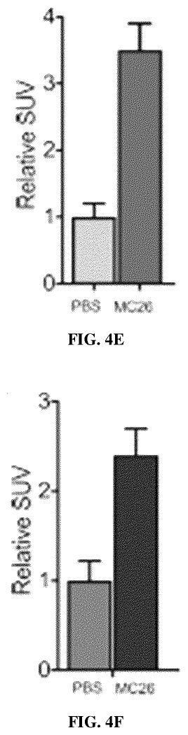

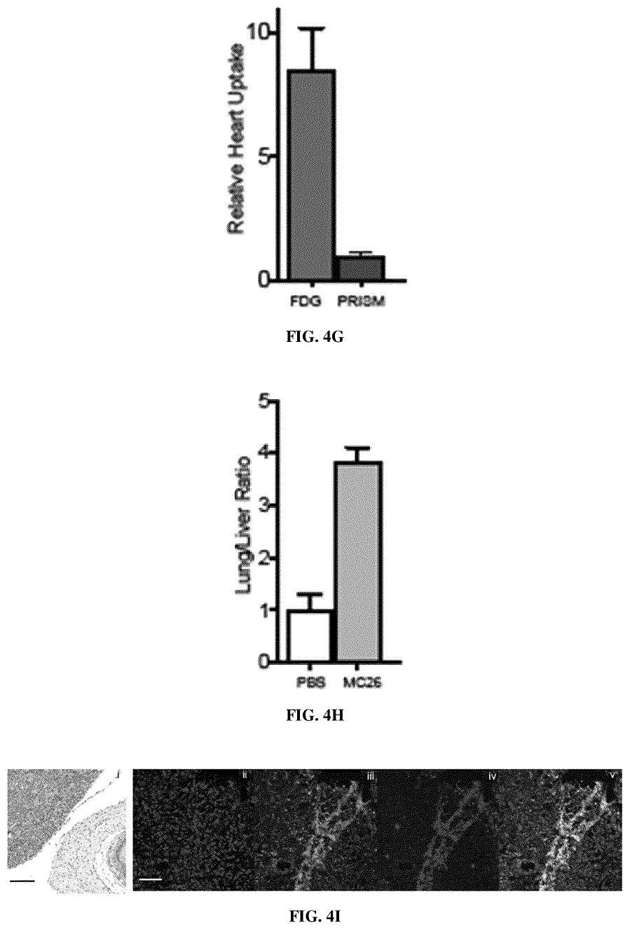

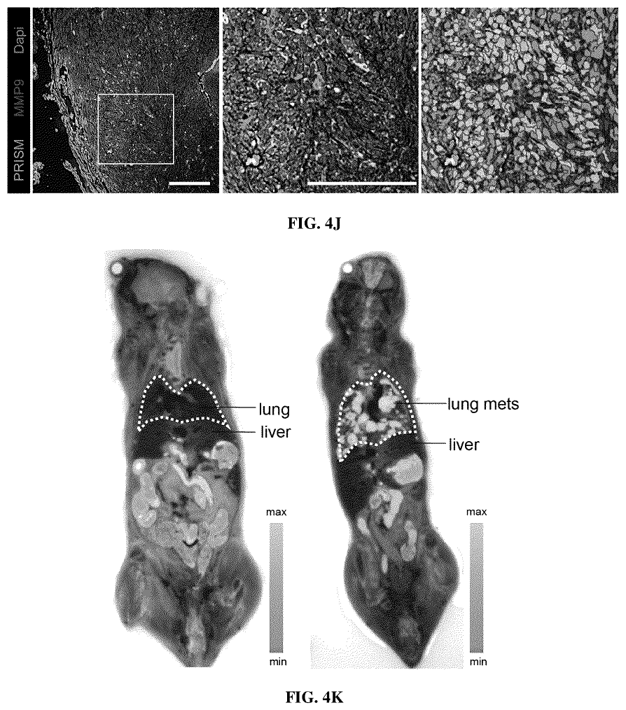

The efficacy of the sensors were also demonstrated in mice bearing CRC lung metastases, which exhibit low glucose uptake tumor (LS174T, data not shown). The CRC lung model was established through intravenous injection of MMP9-secreting MC26 cells in female Balb/c mice. Massive metastasis formation was observed within 2-3 weeks of tumor inoculation (FIG. 4B). Upon intravenous administration of sensors, urine samples from tumor-bearing and healthy control mice were collected after 1 hr, and the ELISA readouts of reporter levels increased as tumor growth progressed (FIG. 4C). Singly-labeled ⁶⁴Cu-PRISM successfully imaged CRC lung metastases, resulting in positive-to-negative tumor ratios of 2.4:1 (FIGS. 4D and 4E). Then the PET/CT images of the same mice were compared, imaged with ⁶⁴Cu-PRISM and ¹⁸F-FDG. Although the standard uptake value is comparable, FDG has a strong accumulation in the heart, and the resulting high background signal obscures covers some of the lung-derived signal, whereas the application of PRISM gave rise to significantly less background signal in the chest (FIGS. 4D, 4F, 4G, and 4H). Therefore, PRISM holds great potential in tracking tumors in certain organs compared with conventional PET tracer. In the CRC lung metastasis model, PRISM also specifically localized to the tumor invasion front, and colocalized with hypoxic regions (FIG. 4I).

### PRISM Monitors Treatment Efficacy in Mouse Models of CRC Metastasis.

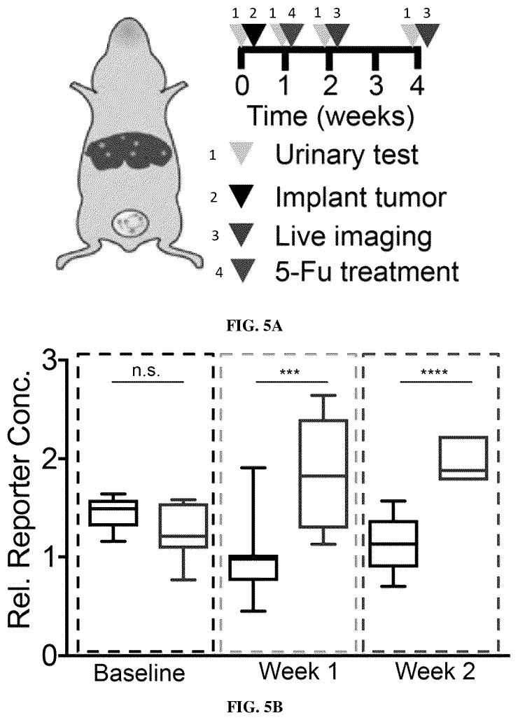

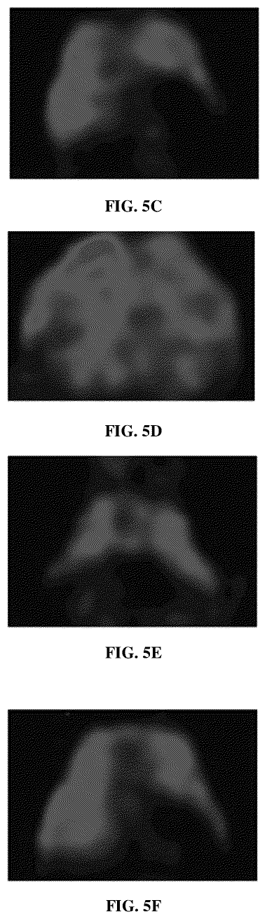

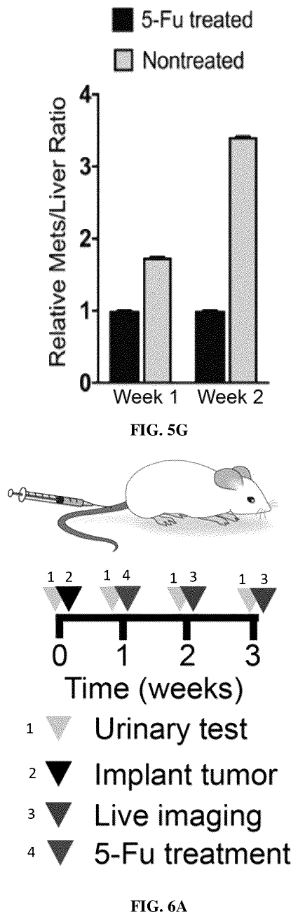

After validation of the efficacy of tailored PRISM nanoparticles, these sensors were applied to study how proteolytic activities correlate with changes in the progression of disease over time and in response to drug treatment. In addition, molecular imaging was safely performed for any lesion for multiple times, providing information related to regional heterogeneity in a given tumor. To evaluate PRISM in monitoring treatment responses, mice were grouped into two categories (n=5 each) to allow monitoring the effect of standard chemotherapy 5-FU treatment: one group received drug (15 mg/kg) by i.p. injection (daily injection for 4 days and once every other day till end of study); and the second group received vehicle as control. To monitor therapeutic responses in treating metastases, PRISM was intravenously injected in each group of mice. After 1 hr of sensor injection, urinary reporter level was assayed to track local proteolytic activity, reflecting tumor invasion capacity. At 6 hrs post-injection, mice were scanned using a small animal PET/CT imager for longitudinal assessment of the tumor progression. Starting 5-FU treatment two weeks after tumor inoculation in the CRC liver metastasis model significantly inhibited tumor progression, consistent with the decreased urinary reporter level compared with the non-treated cohort (FIG. 5B). Real-time images were analyzed and standard uptake value of radioactive reporters were quantified using VivoQuant software. Over 2 weeks of treatment, untreated mice showed substantially increased tumor burden in the liver (FIGS. 5C-5D). In contrast, 5-FU treatment significantly slowed tumor growth compared with the untreated controls (FIGS. 5D-5E).

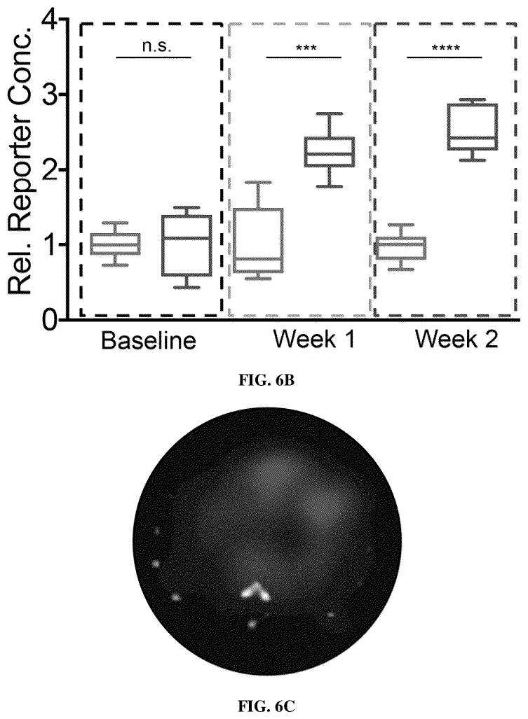

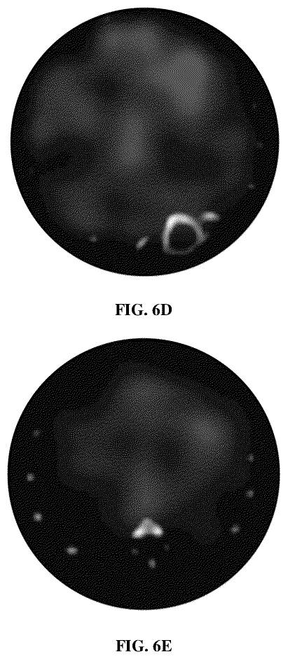

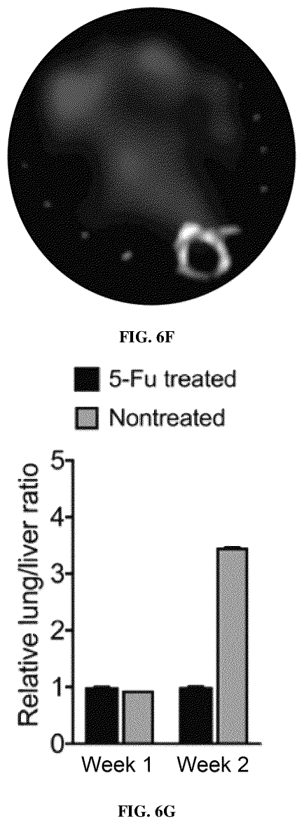

To monitor therapeutic responses when metastases in the CRC lung metastasis model were treated, PRISM was again intravenously injected in two group of mice on a weekly basis. One hour following sensor injection, urinary reporter level was read to track the local proteolytic activity. Significantly reduced urinary reporter levels were observed in 5-FU treated mice, reflecting decreased tumor invasion capacity compared with untreated control group. 6 hours after sensor injection, PET/CT were applied to monitor tumor growth or regression, which provides a longitudinal, noninvasive assessment of the efficacy of anti-tumor drug treatment. Every mouse that received 5-FU showed slower tumor growth compared with the untreated controls. Consistently, the relative lung/liver ratio of PET/CT signal in the cohort that received no treatment was over 3 times higher than that of the cohort treated with chemotherapy (n=5) (FIG. 6G).

Table 5 includes a list of pH low insertion peptides used in this Example. In Table 5, lower case letters indicate a D-form amino acid, “N3” indicates an azide side chain, and “Cy7” indicates cyanine.

TABLE 5
Peptides.

| Name of Peptide | Sequence (N→C) | Type of pHLIP |
|---|---|---|
| pHLIP | AAEQNPIYWARYADWLFTTPLLLLDL | pHLIP |
| | ALLVDADEGTC (SEQ ID NO: 31) | |
| pHLIP-F | AAEQNPIYWARYADWLFTTPLLLLDL | pHLIP with fluorophore |
| | ALLVDADEGTC(Cy7) (SEQ ID NO: 32) | |
| pHLIP-V3 | ACDDQNPWRAYLDLLFPTDTLLLDLL | modified pHLIP (used |
| | Wkk (SEQ ID NO: 33) | in this Example) |
| pHLIP-V3-F | AC(Cy7)DDQNPWRAYLDLLFPTDTLL | modified pHLIP (used |
| | LDLLWkk (SEQ ID NO: 34) | in this Example) |
| pHLIP-V3-L | K(N3)GGACDDQNPWRAYLDLLFPTD | modified pHLIP (used |
| | TLLLDLLWkk (SEQ ID NO: 35) | in this Example) |
| D-pHLIP-V3-L | K(N3)GGacddqnpwrayldllfptdtllldllwkk | modified pHLIP (used |
| | (SEQ ID NO: 36) | in this Example) |
| NT-pHLIP-V3 | ACDDQNPWRAYLKLLFPTKTLLLKLL | non-targeting control |
| | Wkk (SEQ ID NO: 37) | peptide (used in this |
| | | Example) |
| NT-pHLIP-V3-F | AC(Cy7)DDQNPWRAYLKLLFPTKTLL | non-targeting control |
| | LKLLWkk (SEQ ID NO: 38) | peptide (used in this |
| | | Example) |
| NT-pHLIP-V3-L | K(N3)GGACDDQNPWRAYLKLLFPTK | non-targeting control |
| | TLLLKLLWkk (SEQ ID NO: 39) | peptide (used in this |
| | | Example) |
| D-NT-pHLIP-V3-L | K(N3)GGacddqnpwrayldllfptdtllldllwkk | non-targeting control |
| | (SEQ ID NO: 40) | peptide (used in this |
| | | Example) |

### Additional Embodiments

Paragraph 1. A protease imaging sensor comprising:
(a) a scaffold linked to an enzyme-specific substrate that is attached to a first detectable marker, wherein the first detectable marker is capable of being released from the sensor when exposed to an enzyme, and,
(b) a tumor imaging agent comprising a second detectable marker, wherein the tumor imaging agent is linked to the scaffold and wherein the tumor imaging agent does not include a cell penetrating domain.

Paragraph 2. The protease imaging sensor of paragraph 1, wherein the tumor imaging agent further comprises a pH low insertion peptide.

Paragraph 3. The protease imaging sensor of paragraph 1 or 2, wherein the scaffold comprises a protein, a polymer, or a nanoparticle, optionally wherein the protein, polymer or nanoparticle is greater than about 5 nm in diameter.

Paragraph 4. The protease imaging sensor of any one of paragraphs 1 to 3, wherein the scaffold comprises a multi-arm polyethylene glycol molecule (multi-arm PEG), optionally wherein the multi-arm PEG comprises 2-20 arms, optionally wherein the multi-arm PEG comprises 8 arms.

Paragraph 5. The protease imaging sensor of paragraph 4, wherein the multi-arm PEG has a total diameter between 5 nm and 20 nm, optionally wherein the multi-arm PEG has a total diameter of about 15 nm.

Paragraph 6. The protease imaging sensor of any one of paragraphs 1 to 5, wherein the scaffold comprises an iron oxide nanoparticle (IONP), optionally wherein the IONP is between about 10 nm and about 20 nm in size.

Paragraph 7. The protease imaging sensor of any one of paragraphs 1 to 6, wherein each enzyme-specific substrate comprises a cancer substrate, optionally wherein the cancer substrate is cleaved by an enzyme associated with colorectal cancer (CRC).

Paragraph 8. The protease imaging sensor of paragraph 7, wherein the cancer substrate is a cancer metastasis substrate, optionally wherein the cancer metastasis substrate is cleaved by an enzyme associated with colorectal cancer metastasis.

Paragraph 9. The protease imaging sensor of any one of paragraphs 1 to 8, wherein the first detectable marker comprises a peptide, a nucleic acid, a small molecule, a fluorophore, a carbohydrate, a particle, a radiolabel, a MRI-active compound, a ligand encoded reporter, or a isotope coded reporter molecule (iCORE).

Paragraph 10. The protease imaging sensor of paragraph 9, wherein the second detectable marker comprises a radiolabel and is detectable by positron emission tomography or computerized tomography.

Paragraph 11. The protease imaging sensor of paragraph 10, wherein the radiolabel is selected from the group consisting of ⁶⁴Cu, Gd(DOTA), ²⁰¹Tl, ^{99m}Tc, ¹⁸F-2-deoxyfluoroglucose (FDG), (18)F-fluoride, gadodiamide, radioisotopes of Pb(II), ¹¹¹In, and ⁸⁹Zr.

Paragraph 12. The protease imaging sensor of any one of paragraphs 10 or 11, wherein the second detectable marker comprises a metal chelator selected from the group consisting of 1,4,7-Triazacyclononane-1,4,7-triacetic acid (NOTA), 1,4,7,10-tetraazacyclododecane-N,N′,N″,N′″-tetraacetic acid (DOTA), Diethylenetriaminepentaacetic Anhydride (DTPA), 1,4,8,11-tetraazacyclotetradecane-1,4,8,11-tetraacetic acid (TETA), and deferoxamine (for 89Zr).

Paragraph 13. The protease imaging sensor of any one of paragraphs 2 to 12, wherein the pH low insertion peptide is selected from the group consisting of SEQ ID NOs: 14-40.

Paragraph 14. The protease imaging sensor of any one of paragraphs 1 to 13, wherein the wherein the pH low insertion peptide comprises a D-amino acid, an azide side chain, and/or cyanine.

Paragraph 15. The protease imaging sensor of any one of paragraphs 1 to 14, wherein the tumor imaging peptide is N-terminally linked to the scaffold.

Paragraph 16. The protease imaging sensor of any one of paragraphs 1 to 15, wherein the scaffold comprises a single enzyme-specific substrate, a single tumor imaging peptide, or a combination thereof.

Paragraph 17. The protease imaging sensor of any one of paragraphs 1 to 16, wherein the scaffold comprises multiple enzyme-specific substrates, multiple tumor imaging peptides, or a combination thereof.

Paragraph 18. The protease imaging sensor of any one of paragraphs 1 to 17, wherein the ratio of the number of enzyme-specific substrates to the number of tumor imaging peptides is 1:1.

Paragraph 19. A method for detecting a tumor in a subject, the method comprising:
(a) administering to a subject a protease imaging sensor, wherein the protease imaging sensor comprises
   (i) a scaffold linked to an enzyme-specific substrate that is attached to a first detectable marker, wherein the first detectable marker is capable of being released from the sensor when exposed to a cancer-associated enzyme at a site within the subject, and
   (ii) a tumor imaging peptide comprising a second detectable marker, wherein the tumor imaging peptide is linked to the scaffold and wherein the tumor imaging agent does not include a cell penetrating domain; and
(b) detecting in a biological sample obtained from the subject the first detectable marker, wherein detection of the first detectable marker in the biological sample is indicative of the subject having a tumor and/or detecting in the subject the second detectable marker, wherein detection of the second detectable marker indicates the site of exposure to the cancer-associated enzyme.

Paragraph 20. The method of paragraph 19, wherein the tumor imaging peptide comprises a pH low insertion peptide.

Paragraph 21. The method of paragraph 19 or 20, wherein (b) comprises detecting the first detectable marker and detecting the second detectable marker.

Paragraph 22. The method of any one of paragraphs 19 to 21, wherein the biological sample is not derived from the site of exposure to the cancer-associated enzyme, optionally wherein the sample is a urine sample, blood sample, or tissue sample.

Paragraph 23. The method of any one of paragraphs 19 to 22, wherein the site of exposure to the cancer-associated enzyme is a site of metastasis.

Paragraph 24. The method of paragraph 23, wherein the site of metastasis is selected from the group consisting of lung, liver, or heart.

Paragraph 25. The method of any one of paragraphs 19 to 24, further comprising quantifying the amount of the second detectable marker.

Paragraph 26. The method of any one of paragraphs 19 to 25, wherein the detecting of the first detectable marker in (b) comprises a method selected from mass spectrometry, PCR analysis, DNA microarray, fluorescence analysis, a capture assay (e.g., ELISA), optical imaging, magnetic resonance imaging (MRI), positron emission tomography (PET) imaging, computerized tomography (CT) imaging, intraoperative imaging or any combination thereof.

Paragraph 27. The method of any one of paragraphs 19 to 26, wherein the detecting of the second detectable marker in (b) comprises a method selected from fluorescence analysis, optical imaging, magnetic resonance imaging (MRI), positron emission tomography (PET) imaging, computerized tomography (CT) imaging, intraoperative imaging, or any combination thereof.

Paragraph 28. The method of any one of paragraphs 19 to 27, wherein the subject is suspected of having, at risk for, or has cancer, optionally wherein the cancer is colorectal cancer.

Paragraph 29. The method of paragraph 28, wherein the subject has been administered a therapeutic agent.

Paragraph 30. The method of any one of paragraphs 19 to 29, further comprising (c) classifying a cancer as metastatic or non-metastatic.

Paragraph 31. A method for monitoring tumor progression in a subject, the method comprising:
(a) administering to a subject having a tumor a protease imaging sensor, wherein the protease imaging sensor comprises
   (i) a scaffold linked to an enzyme-specific substrate that is attached to a first detectable marker, wherein the first detectable marker is capable of being released from the sensor when exposed to a cancer-associated enzyme at a site within the subject, and
   (ii) a tumor imaging peptide comprising a second detectable marker, wherein the tumor imaging peptide is linked to the scaffold and wherein the tumor imaging agent does not include a cell penetrating domain;
(b) detecting in a biological sample obtained from the subject the first detectable marker, wherein detection of the first detectable marker in the biological sample is indicative of the subject having a tumor and/or detecting in the subject the second detectable marker, wherein detection of the second detectable marker indicates the site of exposure to the cancer-associated enzyme; and
(c) repeating (a) and (b) at least once, thereby monitoring tumor progression in the subject.

Paragraph 32. The method of paragraph 31, wherein the subject has been administered a first therapeutic agent, a first therapeutic intervention has been performed on the subject, or a combination thereof.

Paragraph 33. The method of paragraph 31 or 32, wherein (b) comprises detecting the first detectable marker and detecting the second detectable marker.

Paragraph 34. The method of any one of paragraphs 31 to 33, wherein the tumor imaging peptide comprises a pH low insertion peptide.

Paragraph 35. The method of any one of paragraphs 31 to 34, wherein the biological sample is not derived from the site of exposure to the cancer-associated enzyme, optionally wherein the sample is a urine sample, blood sample, or tissue sample.

Paragraph 36. The method of any one of paragraphs 31 to 35, wherein the site of exposure to the cancer-associated enzyme is a site of metastasis.

Paragraph 37. The method of paragraph 36, wherein the site of metastasis is selected from the group consisting of lung, liver, or heart.

Paragraph 38. The method of any one of paragraphs 31 to 37, further comprising quantifying the amount of the second detectable marker.

Paragraph 39. The method of any one of paragraphs 31 to 38, wherein the detecting of the first detectable marker in (b) comprises a method selected from mass spectrometry, PCR analysis, DNA microarray, fluorescence analysis, a capture assay (e.g., ELISA), optical imaging, magnetic resonance imaging (MRI), positron emission tomography (PET) imaging, computerized tomography (CT) imaging, intraoperative imaging or any combination thereof.

Paragraph 40. The method of any one of paragraphs 31 to 39, wherein the detecting of the second detectable marker in (b) comprises a method selected from fluorescence analysis, optical imaging, magnetic resonance imaging (MRI), positron emission tomography (PET) imaging, computerized tomography (CT) imaging, intraoperative imaging, or any combination thereof.

Paragraph 41. The method of any one of paragraphs 31 to 40, wherein the subject has colorectal cancer.

Paragraph 42. The method of any one of paragraphs 31 to 41, wherein the method further comprises (d) classifying a tumor as progressing, in remission, or stable.

1. A protease imaging sensor comprising:
(a) a scaffold linked to an enzyme-specific substrate that is attached to a first detectable marker, wherein the first detectable marker is capable of being released from the sensor when exposed to an enzyme, and,
(b) a tumor imaging agent comprising a second detectable marker, wherein the tumor imaging agent is linked to the scaffold and wherein the tumor imaging agent does not include a cell penetrating domain.

2.-10. (canceled)

11. A method for detecting a tumor in a subject, the method comprising:
(a) administering to a subject a protease imaging sensor, wherein the protease imaging sensor comprises
(i) a scaffold linked to an enzyme-specific substrate that is attached to a first detectable marker, wherein the first detectable marker is capable of being released from the sensor when exposed to a cancer-associated enzyme at a site within the subject, and
(ii) a tumor imaging peptide comprising a second detectable marker, wherein the tumor imaging peptide is linked to the scaffold and wherein the tumor imaging agent does not include a cell penetrating domain; and
(b) detecting in a biological sample obtained from the subject the first detectable marker, wherein detection of the first detectable marker in the biological sample is indicative of the subject having a tumor and/or detecting in the subject the second detectable marker, wherein detection of the second detectable marker indicates the site of exposure to the cancer-associated enzyme.

12. The method of claim 11, wherein the tumor imaging peptide comprises a pH low insertion peptide.

13. The method of claim 11, wherein the site of exposure to the cancer-associated enzyme is a site of metastasis.

14. The method of claim 11, wherein the detecting of the first detectable marker in (b) comprises a method selected from mass spectrometry, PCR analysis, DNA microarray, fluorescence analysis, a capture assay (e.g., ELISA), optical imaging, magnetic resonance imaging (MRI), positron emission tomography (PET) imaging, computerized tomography (CT) imaging, intraoperative imaging or any combination thereof.

15. The method of claim 11, wherein the detecting of the second detectable marker in (b) comprises a method selected from fluorescence analysis, optical imaging, magnetic resonance imaging (MRI), positron emission tomography (PET) imaging, computerized tomography (CT) imaging, intraoperative imaging, or any combination thereof.

16. The method of claim 11, wherein the subject has been administered a therapeutic agent.

17. A method for monitoring tumor progression in a subject, the method comprising:
(a) administering to a subject having a tumor a protease imaging sensor, wherein the protease imaging sensor comprises
(i) a scaffold linked to an enzyme-specific substrate that is attached to a first detectable marker, wherein the first detectable marker is capable of being released from the sensor when exposed to a cancer-associated enzyme at a site within the subject, and
(ii) a tumor imaging peptide comprising a second detectable marker, wherein the tumor imaging peptide is linked to the scaffold and wherein the tumor imaging agent does not include a cell penetrating domain;
(b) detecting in a biological sample obtained from the subject the first detectable marker, wherein detection of the first detectable marker in the biological sample is indicative of the subject having a tumor and/or detecting in the subject the second detectable marker, wherein detection of the second detectable marker indicates the site of exposure to the cancer-associated enzyme; and
(c) repeating (a) and (b) at least once, thereby monitoring tumor progression in the subject.

18. The method of claim 17, wherein the subject has been administered a first therapeutic agent, a first therapeutic intervention has been performed on the subject, or a combination thereof.

19. The method of claim 17, wherein the tumor imaging peptide comprises a pH low insertion peptide.

20. The method of claim 17, wherein the biological sample is not derived from the site of exposure to the cancer-associated enzyme, optionally wherein the sample is a urine sample, blood sample, or tissue sample.

21. The method of claim 11, wherein the cancer substrate is cleaved by an enzyme associated with colorectal cancer (CRC).

22. The method of claim 21, wherein the cancer substrate is a cancer metastasis substrate.

23. The method of claim 22, wherein the cancer metastasis substrate is cleaved by an enzyme associated with colorectal cancer metastasis

24. The method of claim 11, wherein the second detectable marker comprises a radiolabel and is detectable by positron emission tomography or computerized tomography.

25. The method of claim 24, wherein the radiolabel is selected from the group consisting of ⁶⁴Cu, Gd(DOTA), ²⁰¹Tl, ^{99m}Tc, ¹⁸F-2-deoxyfluoroglucose (FDG), (18)F-fluoride, gadodiamide, radioisotopes of Pb(II), ¹¹¹In, and ⁸⁹Zr.

26. The method of claim 24, wherein the second detectable marker comprises a metal chelator selected from the group consisting of 1,4,7-Triazacyclononane-1,4,7-triacetic acid (NOTA), 1,4,7,10-tetraazacyclododecane-N,N′,N″,N′″-tetraacetic acid (DOTA), Diethylenetriaminepentaacetic Anhydride (DTPA), 1,4,8,11-tetraazacyclotetradecane-1,4,8,11-tetraacetic acid (TETA), and deferoxamine.

27. The method of claim 12, wherein the pH low insertion peptide comprises a sequence that is at least 90% identical to any one of SEQ ID NOs: 14-40.

28. The method of claim 12, wherein the wherein the pH low insertion peptide comprises a D-amino acid, an azide side chain, and/or cyanine.

29. The method of claim 11, wherein the ratio of the number of enzyme-specific substrates to the number of tumor imaging peptides is 1:1.
